(19) United States
(12) Patent Application Publication
(10) Pub. No.: US 2026/0210957 A1
(43) Pub. Date: Jul. 23, 2026

(54) BIOASSAY WITH A LARGE DYNAMIC RANGE BASED ON MICROLASERS

(71) Applicant: The Regents of The University of Michigan, Ann Arbor, MI (US)

(72) Inventors: Xudong FAN, Saline, MI (US); Weishu WU, Ann Arbor, MI (US)

(73) Assignee: The Regents of The University of Michigan, Ann Arbor, MI (US)

(21) Appl. No.: 19/457,481

(22) Filed: Jan. 23, 2026

Related U.S. Application Data
(60) Provisional application No. 63/748,559, filed on Jan. 23, 2025.

(51) Int. Cl. G01N 33/543; B01L 3/00; G01N 33/542; H01S 5/04; H01S 5/42

(52) U.S. Cl. CPC G01N 33/54386; B01L 3/502761; G01N 33/542; B01L 2200/0668; B01L 2300/0654; H01S 5/041; H01S 5/423

(57) ABSTRACT

A bioassay device having a dynamic detection range to quantify at least one biological species includes a region receiving a fluid sample optionally including the biological species. The device includes a plurality of microlasers with at least one optical cavity and a plurality of microunits. Each microunit comprises at least one added lasing energy responsive species (a gain or a lossy medium) to modulate lasing emissions when energy is directed at the microlasers/microunits. Each microunit comprises a targeting component capable of binding with at least one target biological species in the fluid sample. A respective level of lasing emissions generated by the plurality of microunits relates to an amount of the energy directed at the plurality of microlasers and indicates a quantity of the at least one target biological species in the fluid sample. Methods for quantifying at least one target biological species in a fluid sample are also provided.

### CROSS-REFERENCE TO RELATED APPLICATIONS

This application claims the benefit of U.S. Provisional Application No. 63/748,559, filed on Jan. 23, 2025. The entire disclosure of the above application is incorporated herein by reference.

### FIELD

The present disclosure relates to bioassay devices having a large dynamic detection range to quantify at least one biological species and further relates to methods for quantifying at least one target biological species in a fluid sample.

### BACKGROUND

This section provides background information related to the present disclosure which is not necessarily prior art.

There is a rising demand for biological assays (bioassays), due to increasing prevalence of chronic diseases, advancements in biomarker discovery, and need for rapid diagnostic techniques. Bioassays, such as enzyme-labeled or fluorescence-labeled immunoassays, are used to detect and quantify specific soluble substances in a biological sample. These assays are highly selective, however, the detection limit is very narrow and at higher concentrations, they are less accurate. For example, enzyme-linked immunosorbent assay (ELISA) and its variations (such as fluorescence-label immunoassay) are commonly used biological assays (bioassays) that can quantify target species (such as biomolecules) in a sample. In traditional sandwich-type ELISA, capture antibodies are first immobilized on the surface of solid materials (such as glass, metal, or plastics/polymers). After the binding of the target molecules (such as cytokines, interleukin (IL)-6, IL-10, tumor necrosis factor (TNF)), detection antibodies, which are conjugated with enzymes (such as horseradish peroxidase (HRP)), are added to bind with the target molecules bound to the capture antibodies. Finally, substrates are added to interact with enzymes. The enzyme-substrate reaction makes the substrate emit light (such as chemiluminescence or chemifluorescence) or change its color (colorimetric detection). Alternatively, the detection antibodies are conjugated with fluorescent reporters such as dyes and quantum dots, or with plasmonic nanoparticles (i.e., metal nanoparticles), where the emitted light from dyes or quantum dots, or the scattered light from plasmonic nanoparticles are detected to quantify the target analyte. Such detection is called analog detection, since the emitted or scattered light intensity, or the transmitted light (in colorimetric detection) is used to quantify the target analyte. Usually, the detection range is between a few pico-grams per mL to about 10,000 pg/mL (the actual detection range depends on the reagents, assay formats and target analytes).

To further improve the sensitivity (or detection limit), digital ELISA has been developed. For example, one bead-based digital ELISA commercialized by Quanterix uses an enzyme-substrate reaction. Other groups use plasmonic nanoparticles or fluorophores (such as dyes and quantum dots) to replace enzyme-substrate reactions. While digital ELISA can achieve unprecedented sensitivity, it has a very limited dynamic range. For example, the Quanterix digital ELISA can detect cytokines down to 0.01 pg/mL; however, its upper detection limit is only 24 pg/mL. At a higher concentration of target analyte, the basic assumption for digital ELISA, that is, the average number of analytes per bead should be far below 1, breaks down. At a high concentration, the analog detection should be used. To extend the dynamic range beyond digital ELISA, the digital detection signal and the analog signal are stitched together, which may cause detection errors (for example, it is difficult to determine the cut-off (or transition) analyte concentration between the digital detection mode and analog detection mode. A unified method that has a high dynamic range to cover both the lower end of the analyte concentration (such as 0.01 pg/mL) and the upper end of the analyte concentration (such as 10 ng/ml) without any digital-to-analog transition would improve accuracy and be advantageous. These assays are highly selective, however, the detection limit is very narrow and at higher concentrations, they are less accurate. It would be desirable to have highly selective bioassays that are accurate through a wide range of concentrations.

### SUMMARY

This section provides a general summary of the disclosure, and is not a comprehensive disclosure of its full scope or all of its features.

In certain aspects the present disclosure relates to a bioassay device to quantify one or more biological species. The bioassay device may comprise a region for receiving a fluid sample that optionally comprises at least one target biological species. The bioassay device further comprises a plurality of microlasers in contact with at least a portion of the fluid sample. Each microlaser is defined by at least one optical cavity and a microunit capable of generating lasing emissions disposed in the at least one optical cavity. Each microunit comprises at least one added lasing energy responsive species that serves as a gain medium or a lossy medium to modulate lasing emissions. Further, each microunit comprises a targeting component capable of binding with the at least one target biological species in the fluid sample. In this manner, a respective level of lasing emissions generated by the plurality of microlasers relates to an amount of energy directed at the plurality of microlasers and indicates a quantity of the at least one target biological species in the fluid sample.

In one aspect, the at least one optical cavity comprises one or more of a Fabry-Pérot resonator cavity, a distributed feedback based cavity, a photonic crystal based cavity, or a ring resonator.

In one aspect, each microlaser of the plurality of microlasers is a vertical cavity surface emitting laser (VCSEL). Each vertical cavity surface emitting laser (VCSEL) may comprise the microunit that comprises the at least one added lasing energy responsive species and has the targeting component disposed on a surface thereof. In one aspect, the microunit may be a cylindrical semiconductor microunit.

In one aspect, each microunit of each microlaser of the plurality of microlasers is a microparticle formed of a material selected from the group consisting of: polymers, glass, dielectric materials, semiconductor materials, and combinations thereof.

In one aspect, each microunit of each microlaser of the plurality of microlasers is a polymeric bead. The polymeric bead comprises the at least one added lasing energy responsive species. The polymeric bead further defines a surface having the targeting component disposed thereon.

In one aspect, each microunit of each microlaser of the plurality of microlasers is a polymeric bead. The polymeric bead comprises the at least one added lasing energy responsive species. The polymeric bead further defines a surface having the targeting component disposed thereon.

In one aspect, the plurality of microlasers is either (i) mobile and transported through the region for receiving the fluid sample; or (ii) stationary and disposed on a surface within the region for receiving the fluid sample.

In one aspect, the at least one optical cavity is a single optical cavity and the plurality of microlasers is defined by a plurality of microunits disposed with the single optical cavity.

In one aspect, the plurality of microlasers comprises at least 10 microlasers.

In one aspect, the plurality of microlasers comprises at least 1,000 microlasers.

In one aspect, the at least one added lasing energy responsive species is either: (i) the gain medium selected from the group consisting of: a dye, a quantum dot, a semiconductor material, engineered fluorescent molecules, and combinations thereof, or (ii) the lossy medium comprising a lossy molecule or a quenching molecule.

In one aspect, the device further comprises a lasing pump source configured to direct the energy at the plurality of microlasers. The device also comprises a detector configured to receive and detect the lasing emissions from the plurality of microlasers.

In certain further aspects, the present disclosure relates a bioassay device to quantify one or more biological species. The bioassay device may comprise at least one Fabry-Pérot resonator cavity defined between a first reflection surface and a second reflection surface. The at least one Fabry-Pérot resonator cavity is configured to receive a fluid sample that optionally comprises at least one target biological species. The bioassay device further comprises a plurality of microunits disposed in an array within the at least one Fabry-Pérot resonator cavity to define a plurality of microlasers capable of generating lasing emissions. Each microunit of the plurality of microunits comprises at least one added lasing energy responsive species that serves as either a gain medium or a lossy medium that modulates lasing emissions when energy is directed at the plurality of microunits. Each microunit also comprises a targeting component capable of binding with the at least one target biological species in the fluid sample. In this manner, a respective level of lasing emissions generated by the plurality of microlasers relates to an amount of energy directed at the plurality of microunits and indicates a quantity of the at least one target biological species in the fluid sample.

In one aspect, the at least one added lasing energy responsive species is either: (i) the gain medium selected from the group consisting of: a dye, a quantum dot, a semiconductor material, engineered fluorescent molecules, and combinations thereof, or (ii) the lossy medium comprising a lossy molecule or a quenching molecule.

In one aspect, the targeting component is part of an Enzyme Linked ImmunoSorbent Assay (ELISA)-based system that comprises a capture antibody capable of binding with the at least one target biological species. The Enzyme Linked ImmunoSorbent Assay (ELISA)-based system further comprises a detection antibody that comprises an indicator species. The detection antibody further binds to the at least one target biological species that is bound to the capture antibody.

In one further aspect, a first microlaser comprises a first microunit in the plurality of microunits that has at least one target biological species bound to the capture antibody and the detection antibody and produces a first lasing emission at a first level. Further, a second microlaser comprises a second microunit in the plurality of microunits. The second microunit is free of any target biological species bound to the capture antibody and thus produces a second lasing emission at a second level. The second level of second lasing emission is greater than the first level of the first lasing emission.

In one further aspect, the first level relates to a quantity of the at least one target biological species present in the fluid sample.

In one further aspect, the plurality of microunits comprises a plurality of polymeric beads. Each polymeric bead defines a surface comprising the capture antibody or a plurality of the capture antibodies.

In one further aspect, the plurality of polymeric beads comprises polystyrene.

In one further aspect, the plurality of polymeric beads is disposed in the Fabry-Pérot resonator cavity that also includes a metal-enhanced diaminobenzidine (DAB) peroxidase substrate.

In one aspect, the plurality of microunits comprises at least 100 microunits.

In one aspect, the plurality of microunits comprises at least 1,000 microunits.

In one aspect, the bioassay device has a detection range for at least one target biological species at a concentration in the fluid sample of greater than or equal to about 0.01 pg/mL to less than or equal to about 1,000 pg/mL.

In one aspect, the bioassay device further comprises: a lasing pump source configured to direct the energy at the plurality of microunits in the at least one Fabry-Pérot resonator cavity. The bioassay device further includes a detector configured to receive and detect the lasing emissions from the plurality of microlasers.

In certain other aspects, the present disclosure relates to a method of quantifying at least one target biological species in a fluid sample. The method may comprise directing energy from a pump source at a first pump level towards a bioassay device comprising a plurality of microlasers in contact with at least a portion of the fluid sample. The plurality of microlasers is defined by at least one optical cavity and a plurality of microunits capable of generating lasing emissions disposed in the at least one optical cavity. Each microunit comprises at least one added lasing energy responsive species that serves as a gain medium or a lossy medium to modulate lasing emissions and each microunit comprises a targeting component capable of binding with the at least one target biological species in the fluid sample. The method further comprises detecting a first level of lasing emissions generated by the plurality of microlasers at the first pump level. Next, energy is directed from the pump source at a second pump level towards the bioassay device comprising the plurality of microlasers to generate a second level of lasing emissions. The method also comprises detecting the second level of lasing emissions generated by the plurality of microlasers at the second pump level. A concentration of the at least one target biological species in the fluid sample can be determined by comparing the first level of lasing emissions to a first calibration curve at the first pump level and comparing the second level of lasing emissions to a second calibration curve at the second pump level.

In one aspect, the method further comprises determining the concentration in a computer processing unit. Thus, the comparing may comprise using an algorithm to compare the first level of lasing emissions to the first calibration curve and the second level of lasing emission to the second calibration curve. In certain aspects, the algorithm comprises a least squares regression analysis.

In one aspect, the method further comprises determining the concentration of the at least one target biological species within a detection range of greater than or equal to about 10 pg/mL to less than or equal to about 1,000 pg/mL.

In one aspect, the first level of lasing emissions relates to a first lasing fraction of the plurality of microlasers and the second level of lasing emissions relates to a second lasing fraction of the plurality of microlasers. Thus, determining the concentration compares the first lasing fraction to the first calibration curve and the second lasing fraction to the second calibration curve.

In one aspect, the method further comprises changing at least a portion of the fluid sample in the optical cavity in contact with the plurality of microunits between the detecting the first level of lasing emissions generated by the plurality of microlasers at the first pump level and directing energy from the pump source at the second pump level. Thus, the fluid sample is free of the at least one target biological species during either the directing energy from the pump source at the first pump level or the directing energy from the pump source at the second pump level. Further, the first pump level and the second pump level are the same; however, the first level of lasing emissions and the second level of lasing emissions are distinct from one another. In this manner, the concentration of the at least one target biological species when present in the fluid sample can be determined by comparing each respective level of lasing emissions.

In one aspect, the directing energy from the pump source occurs at a plurality of pump levels, including the first pump level, the second pump level, and at least one additional pump level. The detecting occurs at a plurality of levels of lasing emissions generated by the plurality of microlasers, including the first level of lasing emissions, the second level of lasing emissions, and at least one additional level of lasing emissions, wherein the concentration of the at least one target biological species in the fluid sample can be determined by comparing each level of lasing emissions to a corresponding calibration curve at a respective pump level of the plurality of pump levels.

In one aspect, the plurality of pump levels comprise seven distinct pump levels and the plurality of levels of lasing emissions comprise seven distinct levels of lasing emissions.

In one aspect, the bioassay device further comprises at least one region configured to receive the fluid sample that optionally comprises the at least one target biological species. The plurality of microunits is disposed in the at least one region.

In one aspect, the at least one added lasing energy responsive species is either: (i) the gain medium selected from the group consisting of: a dye, a quantum dot, a semiconductor material, engineered fluorescent molecules, and combinations thereof; or (ii) the lossy medium comprising a lossy molecule or a quenching molecule.

In one aspect, the at least one optical cavity comprises a Fabry-Pérot resonator cavity defined between a first reflection surface and a second reflection surface. The Fabry-Pérot resonator cavity is configured to receive the fluid sample that optionally comprises the at least one target biological species. The plurality of microunits is disposed within the Fabry-Pérot resonator cavity.

In one aspect, the targeting component of each microunit capable of binding with the at least one target biological species in the fluid sample is part of an Enzyme Linked ImmunoSorbent Assay (ELISA)-based system. The targeting component comprises a capture antibody capable of binding with the at least one target biological species. The Enzyme Linked ImmunoSorbent Assay (ELISA)-based system further comprises a detection antibody that comprises an indicator species. The detection antibody further binds to the at least one target biological species that is bound to the capture antibody.

In one aspect, the plurality of microunits comprises a plurality of polymeric beads. Each polymeric bead defines a surface comprising at least one of the capture antibody. In certain aspects, the bioassay device further comprises a metal-enhanced diaminobenzidine (DAB) peroxidase substrate.

In one further aspect, the plurality of polymeric beads comprise polystyrene.

In one aspect, the plurality of microunits comprises at least 100 microunits.

In one aspect, the plurality of microunits comprises at least 1,000 microunits.

In yet other aspects, the present disclosure relates to a bioassay device to quantify one or more biological species. The bioassay device comprises a plurality of vertical cavity surface emitting lasers (VCSELs) configured to interact with a fluid sample that optionally comprises at least one target biological species. The plurality of vertical cavity surface emitting lasers (VCSELs) are each associated with a targeting component capable of binding with the at least one target biological species in the fluid sample. A respective level of lasing emissions generated by the plurality of vertical cavity surface emitting lasers (VCSELs) relates to a quantity of the at least one target biological species in the fluid sample.

In one aspect, the at least one added lasing energy responsive species is the gain medium comprising a semiconductor material.

In one aspect, the targeting component is part of an Enzyme Linked ImmunoSorbent Assay (ELISA)-based system and the targeting component comprises a capture antibody capable of binding with the at least one target biological species and the Enzyme Linked ImmunoSorbent Assay (ELISA)-based system further comprises a detection antibody that comprises an indicator species, wherein the detection antibody further binds to the at least one target biological species that is bound to the capture antibody.

In one aspect, a first vertical cavity surface emitting laser (VCSEL) in the plurality of vertical cavity surface emitting lasers (VCSELs) has at least one target biological species bound to the capture antibody and the detection antibody and produces a first lasing emission at a first level. Further, a second vertical cavity surface emitting laser (VCSEL) in the plurality of vertical cavity surface emitting lasers (VCSELs) is free of any target biological species bound to the capture antibody and produces a second lasing emission at a second level. The second level differs from the first level. In one further aspect, the second level may be greater than the first level.

In one aspect, the first level relates to a quantity of the at least one target biological species present in the fluid sample.

In one aspect, each microunit portion of each vertical cavity surface emitting laser (VCSEL) of the plurality of vertical cavity surface emitting lasers (VCSELs) defines a surface comprising the capture antibody or a plurality of the capture antibodies.

In one aspect, the plurality of vertical cavity surface emitting lasers (VCSELs) comprises a plurality of microunits that is disposed in an optical cavity that includes a metal-enhanced diaminobenzidine (DAB) peroxidase substrate.

In one aspect, the plurality of vertical cavity surface emitting lasers (VCSELs) comprises at least 100 vertical cavity surface emitting lasers (VCSELs).

In one aspect, the plurality of vertical cavity surface emitting lasers (VCSELs) comprises at least 1,000 vertical cavity surface emitting lasers (VCSELs).

In one aspect, the bioassay device has a detection range for at least one target biological species at a concentration in the fluid sample of greater than or equal to about 0.01 pg/mL to less than or equal to about 1,000 pg/mL.

In one aspect, the plurality of vertical cavity surface emitting lasers (VCSELs) each comprises a microunit comprising a semiconductor material and the device further comprises:
a lasing pump source configured to direct the energy towards each microunit of the plurality of vertical cavity surface emitting lasers (VCSELs) disposed in at least one optical cavity; and
a detector configured to receive and detect the lasing emissions from the plurality of vertical cavity surface emitting lasers (VCSELs).

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1B show variations of bioassay systems including a bioassay device prepared in accordance with certain aspects of the present disclosure that can detect the presence of and/or quantify one or more biological species. FIG. 1A shows a bioassay system that includes an optical cavity and a plurality of microunits and further having an Enzyme Linked ImmunoSorbent Assay (ELISA)-based detection system to target and bind with the one or more biological species. FIG. 1B shows a specific variation of a bioassay system that includes a Fabry-Pérot resonator cavity and a plurality of microunits in the form of polystyrene microbeads doped with a laser gain medium (such as fluorescent dye) and likewise having an Enzyme Linked ImmunoSorbent Assay (ELISA)-based detection system to target and bind with the one or more biological species.

FIGS. 2A-2F generally show a conceptual illustration of a microlaser ensemble (ME) quenching based bioassay according to certain aspects of the present disclosure. FIG. 2A shows a microlaser is formed by placing a microbead doped with a laser gain medium (such as dye) into a liquid-filled micro Fabry-Pérot resonator cavity (F-P) cavity (liquid medium not shown for clarity). The microlaser emits laser signal (bright microlaser) when the external pump exceeds its lasing threshold. FIG. 2B shows introduction of sufficient quenchers into the microlaser (on or near the surface of the microbead(s)) to cause its lasing threshold to surpass the pumping energy density, thus turning off the laser emission (dark microlaser). FIG. 2C shows a microlaser ensemble, referring to a group of identical doped microbeads deposited in the same F-P cavity. When the external pump exceeds the lasing threshold of a microlaser, all the microlasers in the ensemble emit laser signals (bright microlasers). FIG. 2D shows that when quenchers are introduced into the microlasers, the lasing threshold of each microlaser increases according to the number of quenchers on or near the microbead surface, creating a lasing threshold distribution within the microlaser ensemble. Under the same external pump, some microlasers are quenched (dark microlasers) whereas the rest of microlasers still have laser emission (bright microlasers). FIG. 2E shows an illustration of measuring the lasing threshold distribution in a microlaser ensemble. Quencher-free microlasers have the same lasing thresholds as shown by the d-function-like green line on the left. The presence of quencher distribution in the microlaser ensemble leads to a distribution of the lasing threshold as represented by the blue curve. Under a given pumping energy density, the microlasers in the blue-shaded area, which have the lasing threshold lower than the pumping energy density, have the laser emission (bright microlasers), whereas the microlasers in the gray-shaded area are quenched (dark microlasers). By counting the bright microlasers within the ensemble when the pumping energy density is varied, the lasing threshold distribution can be mapped. FIG. 2F shows mapping of lasing threshold distribution is equivalent to mapping the quencher distribution and hence the analyte distribution within the microlaser ensemble, which is in turn related to the analyte concentration in a solution. The δ-function-like green line on the left represents the situation where none of the microlasers in the ensemble has a quencher on or near the microbead surface.

FIG. 3 is an illustration of a microlaser ensemble (ME) quenching based bioassay according to certain aspects of the present disclosure. For simplicity, only one or two representative microunits are shown but represent a process for the entire ensemble. First, doped microbeads conjugated with capture antibodies are incubated with the sample under test. After the biomarkers are captured, enzyme-linked detection antibodies are added and bound to the microbeads, which are subsequently mixed with substrates. The mixture is deposited into a Fabry-Pérot cavity, where an enzyme-substrate reaction produces precipitations that work as quenchers for the microunits/microlasers, producing a distribution of a lasing threshold. This lasing threshold distribution can be measured by changing an external pumping energy density and subsequently used to calculate an analyte concentration. The image on the top left corner shows laser emission from a doped microbead in an Fabry-Pérot resonator cavity. The image on the bottom left corner shows the laser emission from the microlaser (e.g., doped microbead in an Fabry-Pérot cavity) is quenched.

FIGS. 4A-4B. FIG. 4A shows a change in a lasing fraction within comparable microlaser ensembles quenching based bioassay according to certain aspects of the present disclosure as a function of incubation time. Quenchers formed within the microunits over time gradually increase microlasers' lasing thresholds, thus gradually reducing the lasing fraction in the microlaser ensemble under a fixed pumping energy density. Poly-HRP=1 ng/mL. In FIG. 4B, an example of a bioassay device including a microlaser at various pumping energy densities over various incubation times is shown. Each image has dimensions of 35 μm×35 μm. The rightmost column contains white light images of the microlaser (bead).

FIGS. 5A-5B shows a lasing fraction of a microlaser ensemble bioassay prepared in accordance with certain aspects of the present disclosure with respect to the analyte (streptavidin) concentration under various pumping energy densities. FIG. 5A shows raw data. FIG. 5B shows normalized data to zero analyte concentration for each pumping energy density. Note that in the logarithmic scale in the x-axis, 10⁻⁵ pg/mL is used to represent zero analyte concentration. The logarithmic scale in the x-axis, 10⁻⁵ pg/mL is used to represent zero analyte concentration.

FIGS. 6A-6C. FIG. 6A shows calibration curves obtained using function fitting of lasing fraction versus pumping energy density for the nine sets of data in FIGS. 5A-5B. Each subfigure represents a lasing fraction of a microlaser ensemble (ME) with respect to pumping energy density for a given analyte concentration in solution (C). The function fitting is performed using Eq. (2). μ_{fit} and σ_{fit} refer to the fitted values for the Gaussian distribution. For all nine sets of data, Iₙₒᵣₘ and I₀ are calculated to be 3.4 microjoules/mm² and 1.1 microjoules/mm², respectively. E is set to 1 for the fitting. Note that the above function fitting, the three sets of data from the three highest concentrations (5×10⁶ pg/mL, 1×10⁶, and 2×10⁵ pg/mL) are not included, since their lasing fractions are close to zero, which do not provide enough information for the Gaussian distribution. FIG. 6B shows calibration lines for I_{μ} versus analyte concentration in the log-log scale. FIG. 6C shows 3D fitted surface plot of the lasing fraction of the microlaser ensemble with respect to pumping energy density and analyte (streptavidin) concentration. The surface fitting is shown with scattered experimental data (dots). Here, log(C) refers to the logarithmic analyte concentration in units of pg/mL. Note that some datapoints appear darker than others, meaning that they are below the surface, whereas others are on or above the surface.

FIGS. 7A-7B each show a fraction of lasing beads at different IL-6 concentrations under various pumping powers in a semi-log scale (FIG. 7A) or a log-log scale (FIG. 7B) in accordance with certain aspects of the present disclosure. Various pumping powers are as follows: 25 microjoules/mm² is labeled 1, 45 microjoules/mm² is labeled 2, 68 microjoules/mm² is labeled 3, 90 microjoules/mm² is labeled 4, 125 microjoules/mm² is labeled 5, 250 microjoules/mm² is labeled 6, and 375 microjoules/mm² is labeled 7.

FIGS. 8A-8C. FIG. 8A shows calibration curves obtained using function fitting of lasing fraction versus pumping energy density for the six sets of experimental data. Each subfigure represents the lasing fraction of an ME with respect to pumping energy density for a given analyte concentration in solution (C). The function fitting uses Eq. (2). μ_{fit} and σ_{fit} refer to the fitted values for the Gaussian distribution. For all six sets of data, Iₙₒᵣₘ and I₀ are calculated to be 2.1 microjoules/mm², and I₀ is calculated to be 0.1 microjoules/mm², respectively. E is set to 1 for the fitting. FIG. 8B shows calibration lines for I_{μ} versus analyte concentration in the log-log scale. FIG. 8C shows 3D fitted surface plot of the lasing fraction of the microlaser ensemble with respect to pumping energy density and analyte (IL-6) concentration. The surface fitting is shown with scattered experimental data (dots). Here, log(C) refers to the logarithmic analyte concentration in units of pg/mL. Note that some datapoints appear darker than others, meaning that they are below the surface, whereas others are on or above the surface.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

In this application, including the definitions below, the term “module” or the term “controller” may be replaced with the term “circuit,” for example, when used in the context of a computing device or module, etc. The term “module” and/or “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module and/or controller may include one or more interface circuits. In some examples, the interface circuits may include wired or wireless interfaces that are connected to a local area network (LAN), the Internet, a wide area network (WAN), or combinations thereof. The functionality of any given module and/or controller of the present disclosure may be distributed among multiple modules and/or controllers that are connected via interface circuits. For example, multiple modules and/or controllers may allow load balancing. In a further example, a server (also known as remote, or cloud) module and/or controller may accomplish some functionality on behalf of a client module and/or controller.

The term code, as used above, may include software, firmware, and/or microcode, and may refer to programs, routines, functions, classes, data structures, and/or objects. The term shared processor circuit encompasses a single processor circuit that executes some or all code from multiple modules and/or controllers. The term group processor circuit encompasses a processor circuit that, in combination with additional processor circuits, executes some or all code from one or more modules and/or controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules and/or controllers. The term group memory circuit encompasses a memory circuit that, in combination with additional memories, stores some or all code from one or more modules and/or controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112 (f) unless an element is expressly recited using the phrase “means for,” or in the case of a method claim using the phrases “operation for” or “step for.”

Example embodiments will now be described more fully with reference to the accompanying drawings.

In various aspects, the present disclosure contemplates new bioassay devices and methods to quantify one or more biological species. The device has a region or cavity configured to receive a fluid sample that may comprise one or more target biological species to be detected and quantified by the device. In certain variations, the device has a cavity that is an optical cavity, also known as a resonating cavity or optical resonator or cavity resonator, which includes optical elements that confine electromagnetic waves within a bounded structure or configuration, for example, ring resonators, a Fabry-Pérot cavity, a distributed feedback based cavity, photonic crystal structures, and the like. It will be understood that “optical” as used herein refers to an “electromagnetic wave” that spans the entire electromagnetic spectrum, ranging, for example, from deep ultraviolet to far infrared and microwave wavelengths. In certain variations, the device may have a plurality of optical cavities.

The device also includes a plurality of microunits disposed within the cavity. A “microunit” is a component having at least one dimension on a microscale or nanoscale that is capable of generating lasing emissions. For example, the microunit may form a part of or behave as a laser, such that a stimulated or lasing emission may be generated at a given level in response to receiving energy, such as pulses of lasing energy from an external source. A “microunit” as used herein encompasses a “nanounit,” where the microscale component has at least one dimension of less than about 1,000 micrometers (i.e., 1 mm), optionally less than or equal to about 500 micrometers, and optionally less than or equal to about 100 micrometers. A “nanounit” or “nano-sized” component may have at least one spatial dimension that is less than about 1 micrometer (i.e., 1,000 nm). In various aspects, the present disclosure contemplates a bioassay device that includes a plurality of microlasers, which includes such microunits or nanounits.

Notably, a microunit or nanounit will contain a gain (or lossy) medium, but in certain variations, on its own does not create a laser. Rather, the microunit (or nanounit) can form a laser when incorporated into a resonating cavity or optical resonator, for example having top mirror and parallel bottom mirror in a Fabry-Pérot resonator cavity, so that it can then generate lasing emissions when the microunit and gain (or lossy) medium are stimulated. For example, in certain variations of the present disclosure, a microlaser array or microlaser ensemble, includes multiple microlasers formed by multiple microunits (e.g., microbeads) disposed in a single Fabry-Pérot cavity having relatively large dimensions. Each microlaser is respectively formed by a single microunit (e.g., microbead) and the optical (e.g., Fabry-Pérot resonator) cavity. Theoretically, a Fabry-Pérot resonator cavity can be broken into many small pieces (or many small pairs of mirrors). Each pair of mirrors has one microunit therebetween to form a microlaser.

In various aspects, the bioassay device may include a plurality of microlasers each incorporating such a microunit, where at least one dimension of the cavity (for example, a distance between parallel mirrors) is also on a microscale of less than or equal to about 1,000 micrometers. However, it will be appreciated that dimensions of the laser do not necessarily have to be on a microscale, despite containing the microunit(s) that are on a microscale. Moreover, the bioassay device may comprise a plurality of optical cavities, where in certain variations, each microunit may have its own independent optical cavity. In other variations, all the microunits share the same common large-sized single optical cavity.

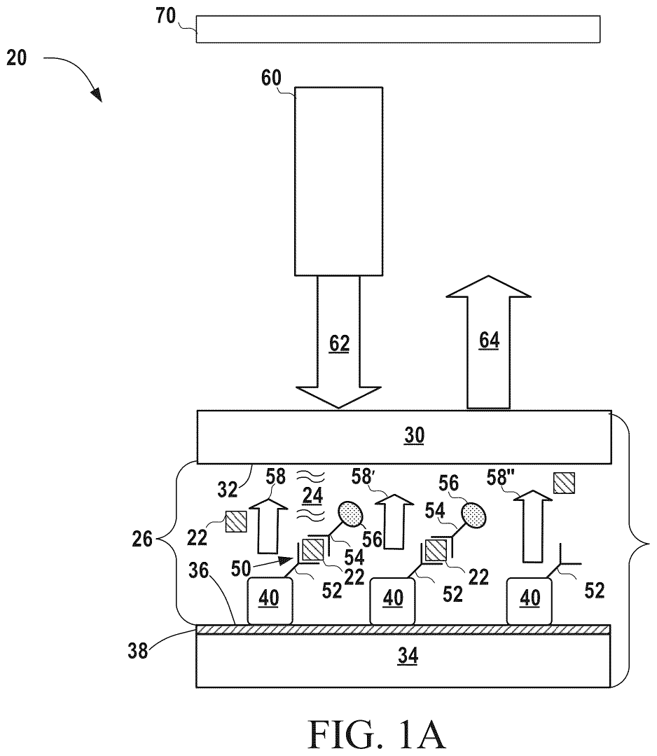

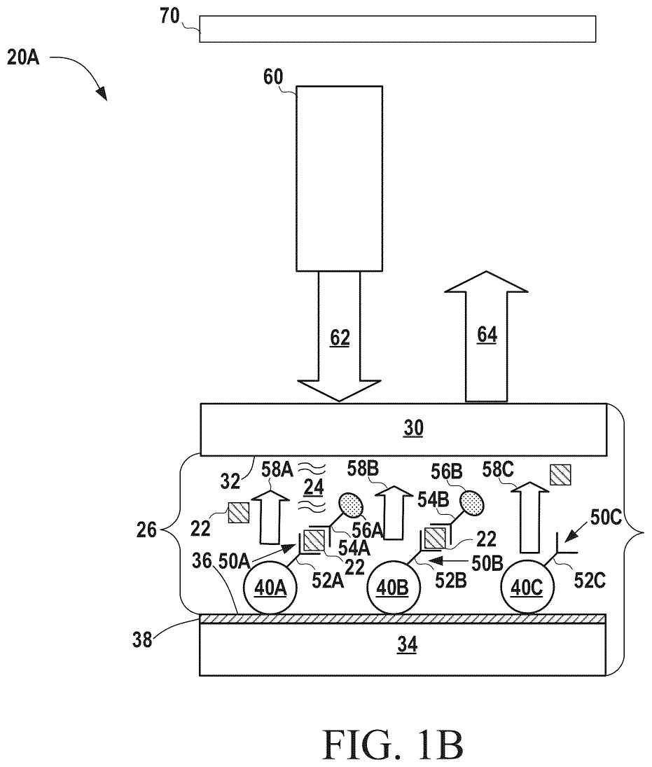

By way of non-limiting example, in one variation, like that shown in FIG. 1A, a microlaser is formed by a microunit (e.g., a microbead that has an average diameter of about 5 micrometers), which is disposed in an optical cavity. The optical cavity may be a Fabry-Pérot cavity that includes two mirrors, a first mirror on top and a second mirror on the bottom of the microbead. The distance between the two mirrors may be 100 micrometers, by way of example. A lateral size of the microunit may be about 5 micrometers by about 5 micrometers. Therefore, the microunit defines a “microlaser” with at least one dimension smaller than 1,000 micrometers (microns).

In certain aspects, the bioassay device comprises a plurality of microunits in the region or cavity that defines an ensemble or array of microunits. In certain aspects, the micro-component may be a microunit. A microunit may be a microparticle. The microunit may define a variety of different shapes, such as a microbead, a microcube/micro cubic, a microcylinder, microrod, and the like, by way of non-limiting example. The microunits can form a microlaser, for example, when they are disposed in a cavity defined by at least two parallel mirrors disposed at the top and bottom of a cavity (to define a Fabry-Pérot cavity). Thus, the plurality of microunits can define the ensemble or array of components that behave as microlasers within the cavity. These microunits also contain at least one added lasing energy responsive species that serves as either a gain medium or a loss medium that modulates lasing emissions generated by the microunit in response to energy being directed at each respective microunit. In this manner, a microlaser, which also encompasses nanolasers, can be formed by the plurality of microunits (e.g., microbeads, cylindrical semiconductor microunits that form part of vertical cavity surface emitting lasers (VCSELs), and the like) disposed within a Fabry-Pérot cavity. A plurality of microunits (e.g., microbeads, cylindrical semiconductor microunits that form part of vertical cavity surface emitting lasers (VCSELs)) disposed within a Fabry-Pérot cavity can thus form a microlaser ensemble.

In addition to the microunits comprising at least one added lasing energy responsive species that serves as either a gain medium or a loss medium that modulates lasing emissions generated by each microunit, each of the plurality of microunits may further comprise a targeting component capable of binding with at least one target biological species in the fluid sample. In this manner, a level of lasing emissions generated by each of the plurality of microunits indicates either an absence or a presence of the at least one target biological species by association with the targeting component.

In various aspects, the plurality of microunits associated with the device may be greater than or equal to about 100 microunits, optionally greater than or equal to about 1,000 microunits, optionally greater than or equal to about 10,000 microunits, optionally greater than or equal to about 100,000 microunits, and in certain variations, optionally greater than or equal to about 1,000,000 microunits. In this manner, as described further below, an entire population of the microunits, for example, hundreds to millions of microunits, can be monitored for emissions generated, where a fraction of the microunits having a bound target analyte may generate lower emissions (or alternatively higher) at a high analyte concentration (at a given pumping power). Likewise, at a lower analyte concentration, on average, fewer analytes are present on, in, and/or near the microunit, so that a fraction of the lasing microunits is higher (or alternatively lower) at the same pumping power. The technology provided by the present disclosure provides bioassay devices with a wide dynamic range of detection capabilities, for example, capable of detecting at least one target biological species at a concentration in the fluid sample of greater than or equal to about 0.01 pg/mL (meaning a lower detection limit of the device goes down to at least about 0.01 pg/mL) to less than or equal to about 1,000 pg/mL (meaning an upper detection limit of the device goes up to at least about 1,000 pg/mL); optionally greater than or equal to about 10 pg/mL to less than or equal to about 1,000 μg/mL.

In various aspects, the microunits may be any known microlaser or nanolaser or those to be developed. For example, the plurality of microunits may include microparticles, including round particles or beads, microcubes/microcubics, microcylinders, ring resonator microunits, disk-like microunits, nanowire lasers, distributed feedback lasers, and/or Fabry-Pérot cavity lasers. In certain aspects, the bioassay device may include a plurality of microunits in the form of cylindrical semiconductor microunits that can define vertical cavity surface emitting lasers (VCSELs). The microunits can be considered to be semiconductor portion of the VCSEL and may have at least one dimension in the microscale or nanoscale. In certain variations, a plurality of microunits, such as cylindrical semiconductor microunits, may be employed to form a plurality of VCSELs described above.

A VCSEL may generally be understood by those of skill in the art to be a semiconductor laser diode formed of multiple layers that generates light emissions that exit the assembly vertically from a surface and thus perpendicular to a stack of layers, as compared to a traditional laser diode where the light exits horizontally from a side that is parallel to the layers. A traditional VCSEL has three parts, a bottom mirror, a cylinder-shaped semiconductor portion, which serves as the gain medium, and a top mirror. The cylinder-shaped semiconductor sits on the bottom mirror and is disposed beneath the top mirror. In a traditional VCSEL, these three parts are in contact without any gap. However, the VSCELs prepared in accordance with certain aspects of the present disclosure include these three components, but the bottom mirror and the cylinder-shaped semiconductor portion are in contact with one another, while the top mirror has a gap between the cylinder-shaped semiconductor portion, so that liquid can fill in or flow through the gap. By way of further background, a VCSEL may include a bottom reflective surface and a top reflective surface, internal to which may be an upper and/or a lower distributed Bragg reflector (DBR). The VCSEL further comprises at least one semiconductor material (as the cylinder-shaped semiconductor portion) that may be considered an added lasing energy responsive species that serves as a gain medium or a lossy medium. Thus, an interior region of the VCSEL may define an active region with one or more quantum wells that may comprise one or more layers and generate photons or lasing emissions by quantum confinement. In certain variations, the semiconductor may be gallium arsenide (GaAs). The quantum wells may include at least three layers of gallium arsenide with different thicknesses. For example, a thin semiconductor (e.g., gallium arsenide) layer may be sandwiched by or disposed between thicker semiconductor (e.g., gallium arsenide) layers. The VCSEL may further have at least one layer for electrical contact on the upper portion of the stack and may have one or more oxide layers disposed on the interior, which can help direct a central beam exiting the VCSEL.

In various aspects, a level of lasing emissions generated by each of the plurality of microunits indicates either an absence or a presence of the at least one target biological species.

In some variations, the plurality of microunits may be formed of a material selected from the group consisting of: polymers, glass, dielectric materials, semiconductor materials, and combinations thereof. The plurality of microunits may be a microparticle formed of a material selected from the group consisting of: polymers, glass, dielectric materials, semiconductor materials, and combinations thereof.

In certain variations, the plurality of microunits may be a polymeric microcomponent (or nanocomponent). For example, a suitable polymeric bead may comprise polystyrene (PS), having at least one dimension on the microscale or nanoscale. By way of example, a microcomponent, such as a microbead, may have a size ranging from greater than or equal to about 0.1 micrometers to less than or equal to about 1,000 micrometers. Each microunit may include at least one added lasing energy responsive species that is responsive to incoming energy directed towards the microunit, which as described below serves to augment modulation of lasing emissions from the microunit when the microunit is associated with a target biological analyte. For example, the microunit may include gain molecules, such as fluorescent dyes, where notably, fluorescent dyes are considered lasing dyes when used in lasers, quantum dots, and engineered fluorescent molecules, or alternatively may include lossy molecules or quenching molecules. In certain aspects, the at least one added lasing energy responsive species is a gain medium and may be selected from the group consisting of: a dye, such as a fluorescent dye/lasing dye, a quantum dot, a semiconductor material, such as gallium arsenide (GaAs) or indium phosphide (InP), engineered fluorescent molecules, and combinations thereof. In other aspects, the at least one added lasing energy responsive species is the lossy medium comprising a lossy molecule or a quenching molecule. In certain aspects, the at least one added lasing energy responsive species is a lossy medium, for example, a quenching dye and/or an enzyme-reactive quenching substrate molecule. The lossy material may be a dye that serves as a quencher, for example, Black Hole Quencher (BHQ) dyes, and enzyme-reactive substrate molecules, such as metal-enhanced diaminobenzidine (DAB). After the reaction with enzyme (such as HRP: Horseradish Peroxidase), the substrate molecules turn into quenching products. For example, in one variation of the present disclosure, HRP and a metal-enhanced DAB are used to generate a quencher.

Furthermore, each of the microunits has at least one targeting component capable of binding with at least one target biological species in the fluid sample. In various aspects, each of the microunits may have a plurality of targeting components capable of associating with the target biological analyte, for example, having a plurality of targeting components disposed on a surface of the microunit that is exposed to the fluid sample that potentially contains the target biological analyte(s). In other alternative aspects, where the bioassay device may include a plurality of vertical cavity surface emitting lasers (VCSELs). Each VCSEL may be associated with a targeting component for binding to any target biological analytes that may be present. In this manner, VCSELs can be used to quantify target biological analytes like the other variations described herein.

In certain aspects, the plurality of microunits are stationary within the cavity. For example, the microunits may be disposed on a surface within the cavity where the fluid sample can be contained or flows through. The microunits, such as microbeads or the microcylinder semiconductor portion of VCSELs, may be disposed and fixed inside a Fabry-Pérot cavity, by way of non-limiting example. In one variation, microbeads can form a ring resonator, where the microbeads are microlasers when they are pumped above the lasing threshold. Such microbeads used as a ring resonator can serve as microlasers that are mobile. The lasing emission goes around the circumference of a bead equator. In other variations, like that shown in FIG. 1B, the microunits are microbeads, which are microsized units that contain gain medium. These microbeads by themselves are not microlasers, but rather are placed inside a Fabry-Pérot cavity. The laser emission(s) thus goes through each microbead body and are perpendicular to the Fabry-Pérot mirror surface. In this variation, the design in the cavity may ensure that fluid flows over and around portions of the microunits, such that gaps or openings between various components permit the flow of fluids over and therebetween.

In other aspects, the plurality of microunits may be mobile and transported through the cavity. In one example, microbeads can flow through a detection point within the cavity, similar to a flow cytometer.

With reference to FIG. 1A, in certain variations, a bioassay system 20 can detect the presence of and/or quantify one or more biological species in the form of one or more biological species or analytes 22. The one or more biological species analytes 22 may be contained in a fluid sample 24 that passes through or is contained within an open region 26 of a cavity 28. The one or more biological species analytes 22 may be soluble in the fluid sample 24. In the variation shown, the cavity 28 may be an optical cavity, such as a Fabry-Pérot resonator cavity 28 specifically, defined between a first support 30 (e.g., layer or substrate) defining a first reflective surface 32 and a second support 34 (e.g., layer or substrate) that may define a second reflective surface 36. The first support 30 may be a transmissive substrate having the first reflective surface 32 on one side. In the variation shown, the second reflective surface 36 may be a surface layer 38 is disposed on the second support 34 facing the first reflective surface 32. The first and second reflective surfaces 32, 36 may be formed by applying a reflective material as a coating over a substrate or support, such as fused silica. Such reflective surfaces 32, 36 may be mirrors. Together, the first reflective surface 32 and the second reflective surface 36 form a pair of parallel reflective surfaces that sandwich the open region 26 of Fabry-Pérot resonator cavity 28 having the fluid sample 24 disposed therein or that flows therethrough.

The optical cavity 28 is configured to receive the fluid sample 24 that optionally comprises one or more target biological species or biological analytes 22. A plurality of microunits 40 (shown generically in FIG. 1A, but not limited to the shapes shown, which may be e.g., microparticles of any shape, round microbeads, the cylinder-shaped semiconductor portion of the vertical cavity surface emitting lasers (VCSELs), and the like) may be disposed on or near (e.g., floating) the surface layer 38 that defines the second reflective surface 36 over second support 34. As will be described herein, the surface layer 38 may increase contrast of any lasing emissions generated by the microunits 40, for example, a metal-enhanced diaminobenzidine (DAB) peroxidase substrate. Metal-enhanced DAB is a lossy medium. However, any loss medium would be suitable, including diaminobenzidine (DAB) peroxidase lacking the metal-enhancement, as well as any other lossy medium like an optical absorber. Notably, a substrate used herein may refer to substrate molecules that react with enzyme molecules to produce quenching molecules. In certain aspects, the substrate molecules may be included in the fluid sample 24 and fill the open region 26 of optical cavity 28 between the first reflective surface 32 and the second reflective surface 36 (e.g., between the two mirrors). In certain aspects, the plurality of microunits 40 may be distributed on or near the surface layer 38 over the second support 34 in an array or regular pattern at predetermined intervals from one another. In other alternative aspects, the microunits 40 may be randomly or unevenly distributed on the surface layer 38.

While not shown in FIG. 1A, in yet other variations, the microunits 40 may not be fixed to a substrate (second support 34) but rather may be free in the fluid sample (e.g., floating in fluid sample 24). Each microunit 40 may comprise at least one added lasing energy responsive species, which serves as either a gain medium or a lossy medium that modulates lasing emissions when energy, such as pulsed laser energy, is directed at the respective microunit 40. Further, each of the plurality of microunits 40 comprises a targeting component 50 capable of associating, reacting, and/or binding with the at least one target biological species analytes 22 in the fluid sample 24. In this manner and as further described below, a respective level of lasing emissions generated by each of the plurality of microunits 40 indicates either an absence or a presence of the at least one target biological species.

In one example, the targeting component 50 is part of an Enzyme Linked ImmunoSorbent Assay (ELISA)-based system. As will be appreciated, many different configurations of ELISA-based detection systems may be employed and the targeting component 50 may be any of the various ELISA-based components used for detecting the presence of biological species analytes 22 known to those of skill in the art. In the embodiment shown in FIG. 1, the targeting component 50 includes a first detection species, for example, a capture antibody 52 capable of binding with the at least one target biological species analytes 22. The targeting component 50 may further include additional detection species, such as a second detection species. For example, the second detection species may be a detection antibody 54 that further comprises an indicator species 56. The detection antibody 54 is applied after the capture antibody 52 binds to the at least one target biological species analytes 22. For example, the second detection species, namely the detection antibody 54 and the indicator species 56, may be applied after the target biological species analytes 22 is bound to the first detection species/capture antibody 52 by applying a substrate solution to which a surface of the microunits 40 is exposed. The indicator species 56 may be an enzyme (e.g., reporter enzyme), such as horseradish peroxidase (HRP) or other known indicator enzymes, by way of non-limiting example. As can be seen, the detection antibody 54 further binds to the at least one target biological species analytes 22 that was previously bound to the capture antibody 54.

It should be noted that incorporation of an enzymatic reaction, for example, by using Enzyme Linked ImmunoSorbent Assay (ELISA)-based system may be optional in certain variations. By incorporating a system that uses enzymatic reactions, it makes detection of the biological analytes or species analytes 22 easier because it chemically increases the number of labeling molecules. However, the devices and methods of the present disclosure may also be used without enzymatic reaction, as well.

The bioassay system 20 may further include a source 60 of electromagnetic radiation or energy 62 that may be directed towards the cavity 28 (e.g., Fabry-Pérot resonator cavity) through the first support 30. The source 60 may be an external optical pumping system, such as a lasing pump, for example. However, in addition to optical pumping, electrical pumping and other types of pump mechanisms can also be used. Laser pumping is the act of energy transfer from an external source into a gain medium (or lossy medium) of a laser, in this case, a gain medium in the microunit 40. The energy may be absorbed in the gain medium (or lossy medium) that produces an excited state of the atoms. When the energy falls from the excited state to ground state, light is emitted. The lasing emissions typically occur over a lasing threshold for the gain medium (or quenching for lossy medium). Laser pumping may be achieved optically by using a pump light source emitting excitation light at the range of wavelengths corresponding to an absorption band or bands of the lasing energy responsive species present in the microunits, such as a gain medium (or lossy medium) in the lasing microunits. Available pumping sources 60 include nanosecond lasers, femtosecond lasers, pulsed or continuous-wave lasers, flashlight, and tunable optical parametric oscillator sources, such as pulsed optical parametric oscillators (OPO). The lasing pump source 60 may generate coherent electromagnetic radiation or energy, which is highly directional. Alternatively, pumping may be possible by fluorescent resonance energy transfer, electrically by injection current, bioluminescence or chemiluminescence. For pulsed laser, a pulse rate can range from a single pulse to a few thousand pulses per second. The pump wavelength depends on the absorption band of the fluorophore or lasing energy responsive species. In certain variations, the lasing threshold of the lasing energy responsive species (e.g., fluorophore or dye) may be from about 0.1 to about 1,000 microjoules per millimeter squared ((μJ)/mm²).

A laser system can thus include three elements, namely a pump source, a gain medium (or lossy medium), and an optical cavity (e.g., a resonator). The optical or resonator cavity 28 may be the Fabry-Pérot resonator cavity described above that is created between the first reflection surface 32 and second reflection surface 36. The microunit 40 comprising at least one lasing energy responsive species may serve as a gain medium that generally amplifies light or alternatively as a lossy medium that dampens or diminishes light as energy is directed thereto from the laser pump/source 60. In certain variations, at least one of the second reflection surface 36 (which may have surface layer 38) or the lasing energy source 60 may be translatable with respect to the microunits 40 in the cavity 28. In certain alternative variations, the microunits may themselves be microcomponents, such as microscale vertical cavity surface emitting lasers (VCSELs) disposed in cavity 28.

The optical cavity 28 is capable of transmitting a portion of a spectrum of electromagnetic radiation 62 from source 60, such that a portion of electromagnetic radiation 62 enters through the first support 30. In the embodiments where the optical cavity 28 is a Fabry-Pérot interference filter (e.g., where the microunits are polymeric microbeads) it may have a configuration that is designed to be a transmission-type cavity, a reflection-type cavity, or a transmission and reflection-type cavity. A reflection-type Fabry-Pérot resonator cavity shows that the portion of electromagnetic energy 62 that enters the open portion of the cavity 28 resonates due to internal reflection between the pair of parallel first and second reflective surfaces 32, 36 serving as a Fabry-Pérot based etalon interference filter. A portion of the electromagnetic energy 62 resonates within the Fabry-Pérot resonator cavity 28 and may be transmitted through the first reflective surface 32 and first support 30. Notably, the portion of electromagnetic energy 62 that enters and resonates in the Fabry-Pérot resonator cavity 28 interacts with each of the microunits 40 present on or near the surface layer 38 of second support 34 to generate lasing emissions, for example, shown as a collective lasing emission output 64, where each microunit may generate a lasing emission 58. As shown, a first lasing emission 58′ is at first level where the microunit 40 has an analyte 22 bound to the targeting component 50, including antibody 52 bound to the analyte 22, as well as to the detection antibody 54 and the indicator species 56, whereas a second lasing emission 58″ is at the second level where no analyte 22 is bound to the targeting component 50, which exit the Fabry-Pérot resonator cavity 28 and cumulatively contribute to the output 64.

Further, the optical cavity 28 in the form of a Fabry-Pérot resonator cavity defined between the first and second reflection surfaces 32, 36 can use a plasmonic effect to enhance a local light-matter interaction and receive the pumping light more efficiently. The resonant wavelength or range of wavelengths of the laser cavity can be selected to overlap with the emission band or bands of lasing energy responsive species used as the gain medium or alternatively, the loss medium.

The bioassay system 20 further includes a detector 70 for receiving and detecting lasing emissions output 64 generated by the microunits 40 (cumulatively lasing emission 58 that includes both first and second lasing emissions 58′ and 58″) within the Fabry-Pérot resonator cavity 28. The detector 70 may be a photodetector or/and spectro-imaging system. Photodetectors may include a charge coupled device (CCD) imager, a CMOS imager, a photo-multiplier tube (PMT), an avalanche photodiode (APD), and the like. The spectro-imaging system may include a charge coupled device (CCD) imager, a spectrometer, combinations thereof, and the like. Such detectors may provide output to a processor, such as a computer processing unit, for example.

Where the detector 70 is a photodetector, it can receive and detect one or more emissions (e.g., laser emissions 58) generated by the at least one lasing energy responsive species in each microunit 40 within the cavity 28.

A specific embodiment of a bioassay system 20A shown in FIG. 1B is like that of bioassay system 20 in FIG. 1A, where the microunits 40A are in the form of round microbeads, such as polymeric beads, like polystyrene (PS) beads. To the extent that the components in the bioassay system 20A of FIG. 1B are the same as bioassay system 20 of FIG. 1B, unless further discussed, they can be assumed to have the same design and/or function in the same manner and thus will not be further discussed herein. Each microunit/microbead 40A can generate its own lasing emission 58A when an external source of energy is pumped towards the Fabry-Perot resonator cavity 28A. Microunit/microbead 40A can include targeting component 50A, which includes capture antibody 52A, as well as to its detection antibody 54A and indicator species 56A. For illustrative purposes, a first microunit or lasing bead 40B has at least one target biological species analyte 22 bound to its capture antibody 52B of its targeting component 50B, as well as to its detection antibody 54B and indicator species 56B. When an external source 60 of energy 62 is pumped towards the Fabry-Perot resonator cavity 28A, the first lasing bead 40B produces a first lasing emission 58B at a first level. Further, a second microunit or lasing bead 40C has no target biological species analytes 22 bound to its targeting component 50C that includes capture antibody 52C (and no second detection species bound, including no detection antibody 54A or indicator species 56A) and produces a second lasing emission 58C at a second level. Depending on whether a lasing energy responsive species included in the first and second lasing beads 40B, 40C is a gain molecule or a lossy molecule, the second level of second lasing emission 58C is either (i) greater than the first level of the first lasing emission 58B in the circumstance where the lasing energy responsive species is a gain molecule or (ii) the second level is less than the first level, where the lasing energy responsive species is a lossy molecule. In various aspects and as will be described further below, the first level of first lasing emission 58B from the first lasing bead 40B relates to a quantity of the at least one target biological species analytes 22 present in the fluid sample 24.

By way of further explanation, the mechanism by which the bioassay system 20 using microunits 40 in FIG. 1A or bioassay system 20A using microbeads 40A in FIG. 1B operates to detect one or more biological species/analytes 22 is further discussed herein. As noted above, the microunits (e.g., micron-sized polystyrene lasing microbeads) 40, 40A may be doped with a laser gain medium (such as dyes, like fluorescent dye, semiconductors) and are placed in an optical cavity 28 or specifically in Fabry-Pérot resonator cavity 28A. One or more exposed surfaces of the microunits 40, lasing beads 40A are coated with the capture antibodies (such as anti-IL-6). Similar to the traditional ELISA method, after the binding of the target analyte 22 (such as IL-6), the detection antibodies (54, 54A) coated with HRP (indicator species 56, 56A) are added. These microunits 40/beads 40A are placed in the optical cavity 28 or Fabry-Pérot resonator cavity 28A, and then substrate solution is added (into fluid 24) and incubated with the microunits 40, beads 40A. Meanwhile, an external pumping source 60 (such as the optical pulses produced by a pulsed laser) is used to excite the dye in the microunit 40, bead 40A making them lase/generate lasing emissions. In the ELISA-based detection system shown in this variation, the lasing from each microunit 40, bead 40A is quenched via horseradish peroxidase (HRP)-substrate reaction, when a substrate solution (e.g., comprising the second detection species, such as a detection antibody 54, 54A with the indicator species 56, 56A).

In the absence of a target analyte (e.g., target biological species 22) on the microunit 40/bead 40A, no detection antibody 54, 54A and indicator species 56 (HRP) are present on the microunit 40, bead 40A surface. Therefore, this microunit 40, bead 40A can lase when it is pumped at a predetermined pumping power (also referred to as a lasing threshold) or higher. However, the presence of the analyte or analytes (target biological species analyte 22) on the surface of the microunit 40, bead 40A results in the presence of HRP molecules on its surface. The reaction of HRP and the substrate (such as metal-enhanced DAB substrate) leads to precipitation around the microunit 40, bead 40A that increases cavity loss for the microunit 40, lasing bead 40A and increases the lasing threshold or even quenches the laser. The more target biological species analytes on the microunit/bead surface, the more precipitation can be generated, and the more difficult for the microunit 40, bead 40A to lase (e.g., generate lasing emissions).

In various aspects, the present methods and device exploit the nonlinear behavior of lasing emission. At a higher analyte concentration in the fluid sample 24, on average, there are more target biological species analytes 22 on each microunit 40, bead 40A. If an entire population of the microunits 40, beads 40A (for example, thousands to millions) are examined, a fraction of the lasing beads is lower (at a given pumping power) for a higher analyte concentration. Likewise, at a lower analyte concentration, on average, fewer target biological species analyte 22 are present on each microunit 40, bead 40A and the fraction of the lasing microunits 40, beads 40A generating lasing emissions is higher (at the same pumping power).

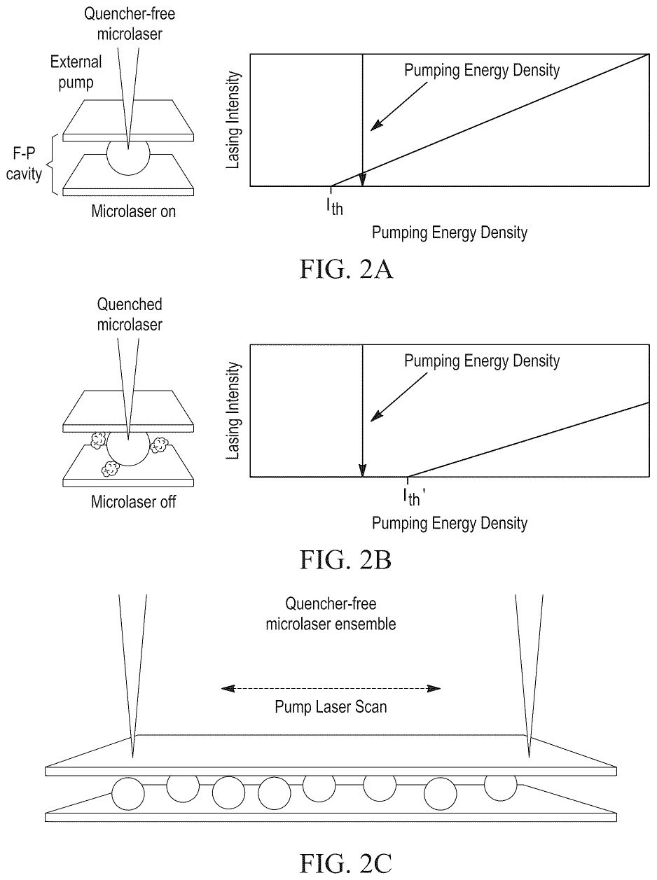

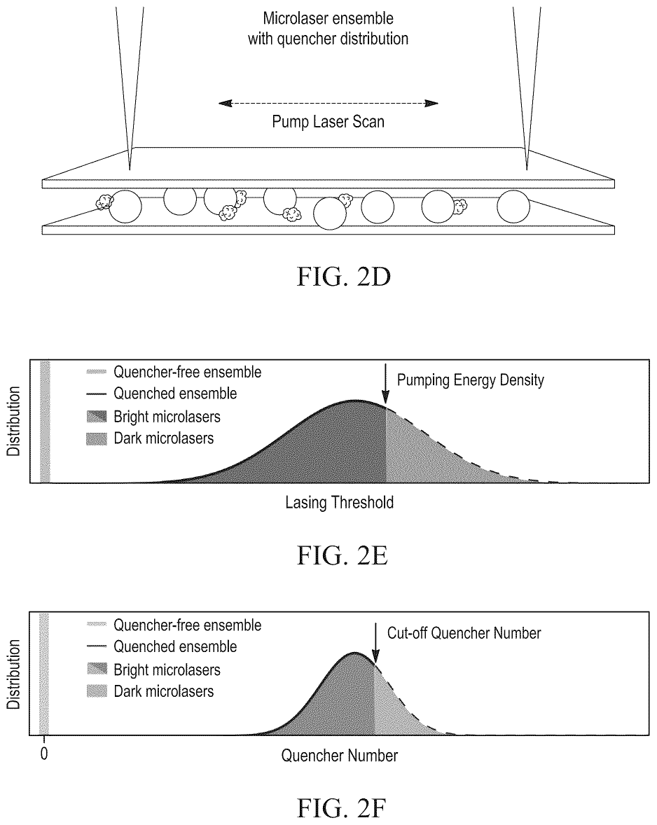

A conceptual illustration of a microlaser ensemble quenching based bioassay according to certain aspects of the present disclosure is shown in FIGS. 2A-2F.

In FIG. 2A, a microlaser is formed by placing a microbead doped with a laser gain medium (such as a dye) into a liquid-filled micro Fabry-Pérot resonator cavity (F-P) cavity (liquid medium not shown for clarity). Each microbead, together with the Fabry-Pérot cavity, is treated as an individual detection microunit. As noted above, other forms of the microunits, such as ring resonator lasers, nanowire lasers, and vertical-cavity surface-emitting lasers (VCSELs) can also be used. A single microlaser is formed by a dye-doped microbead in a Fabry-Pérot cavity, such that when a microlaser is pumped by an external laser at an energy density higher than its lasing threshold, it emits laser signals or lasing emissions. It is referred to herein as a bright microlaser, as illustrated in FIG. 2A. For the same microlaser, when it captures analytes, which results in light absorbing molecules (quenchers) to be near or on the microbead surface (through a process such as enzyme-substrate reaction), the lasing threshold increases. When the lasing threshold surpasses the external pumping energy density, the microlaser is quenched (dark microlaser), as illustrated in FIG. 2B.

In various aspects, the present disclosure contemplates a bioassay device having a plurality of microunits, also referred to herein as a microlaser array or ensemble (ME). The microunits may all have the same composition. In a quencher-free state, when an ME bioassay device is pumped at an energy density slightly higher than its lasing threshold, all microlasers are bright as shown in FIG. 2C. When the bioassay device ME goes through analyte-capturing and quencher-generating processes, the randomness of the analyte binding to the microbeads leads to a distribution of the analytes on the microlasers, which in turn causes a distribution of quenchers and hence lasing thresholds within the ME. Under a given external pumping energy density, only a portion of the microlasers, whose lasing thresholds are lower than the pumping energy density, remain bright, as shown in FIG. 2D.

FIG. 2E presents schematically a distribution of the lasing threshold for a bioassay device as described above. For the case of FIG. 2C (quencher-free), a lasing threshold for all microlasers is the same and low, as represented by the delta (d)-function like green line at 0 on the left. Correspondingly, the number of quenchers on the microlaser is zero and its distribution is a d function (the green line in FIG. 2F). For the case of FIG. 2D, the initial d-function like threshold distribution in FIG. 2E starts to increase and spread. The microlasers whose lasing thresholds are on the left side of the distribution curve (i.e., the lasing thresholds are lower than the pumping energy density) remain bright, whereas those on the right side (i.e., the lasing thresholds are higher than the pumping energy density) become dark (or quenched). By changing the external pumping energy density and counting the fraction of the bright microlasers (which is the cumulative distribution of the lasing threshold), a distribution of the lasing threshold can be mapped and hence the underlying distribution of quenchers on the microlasers shown in FIG. 2F. With the known quencher distribution, the analyte distribution on the microlasers can be recovered and hence the analyte concentration in solution can be determined through a statistical model.

A microlaser quenching equation (MQE) is developed for the ME to study the relationship between the lasing thresholds of microlasers and the number of analytes that they capture. The MQE, as shown in Eq. (1), is built on well-recognized theoretical models for microlasers and an assumption that the quencher production rate is proportional to the number of enzymes, which is in turn proportional to the number of the analytes captured by the microlasers. The equation is:

$${k}_{j} = \frac{ln ( \frac{{I}_{t h ( j )}^{e x p} + {I}_{norm}}{{I}_{0} + {I}_{norm}} )}{E} \qquad ( 1 )$$

states that for the jₜₕ microlaser in the ensemble with the quencher-free lasing threshold, I₀, its experimentally measured lasing threshold,

$${I}_{th ( j )}^{e x p} ,$$

can be used to estimate the number of analytes kⱼ. E, I₀, and Iₙₒᵣₘ are the three fitting parameters determined by the experimental setup.

When the ME bioassay device is exposed to the analytes in solution, the analyte molecules bind to the microunits/microlasers through interactions such as antigen-antibody binding. The binding processes are affected by the random movement of the analyte molecules, leading to a distribution of the analytes on the microlasers, which can be experimentally measured in the following way.

When the ME is pumped at multiple pumping energy densities, a data pair

$$[ ( Lf ( i ) , {I}_{p u m p}^{exp} ( i ) ) ]$$

can be obtained, where Lf(i) is the lasing fraction of the ME at the pumping energy density of

$${I}_{p u m p}^{exp} ( i )$$

As discussed above, a lasing fraction is equivalent to a fraction of microunits/microlasers with the number of analytes smaller than a specific value.

As described further herein, in experiments, the number of microunits/microlasers is always lower than the number of analytes in the test sample, that is, the average number of analytes per microunit/microlaser is larger than one. From the central limit theorem, it is inferred that a distribution of the analytes on the microlasers follows a Gaussian distribution. Therefore, from Eq. (1), the lasing fraction of the ME, Lf(i), and its paired measured pumping energy density

$${I}_{p u m p}^{exp} ( i ) ,$$

should follow a Gaussian cumulative distribution:

$$L f ( i ) = {ϕ}_{c u m u} ( \frac{ln ( \frac{{I}_{p u m p}^{exp} ( i ) + {I}_{norm}}{{I}_{0} + {I}_{norm}} )}{E} ; μ , σ ) , \qquad ( 2 )$$

where φ_{cumu} is the cumulative Gaussian distribution function. μ and σ are the mean and standard deviation for this Gaussian distribution, respectively, which can be obtained via function fitting. μ is related to an analyte concentration in solution. Such a relationship can be established by calibration curves obtained experimentally, which will be discussed below.

Probing the distribution of the analyte on microunits/microlasers (or, more specifically, on microbeads inside the micro Fabry-Pérot cavity) can also be accomplished with simple fluorescence intensity measurements rather than using a microlaser method (or lasing threshold analysis). However, the lasing threshold analysis method is also contemplated in that it is much more sensitive and accurate in obtaining the analyte distribution information among the microbeads.

### Examples

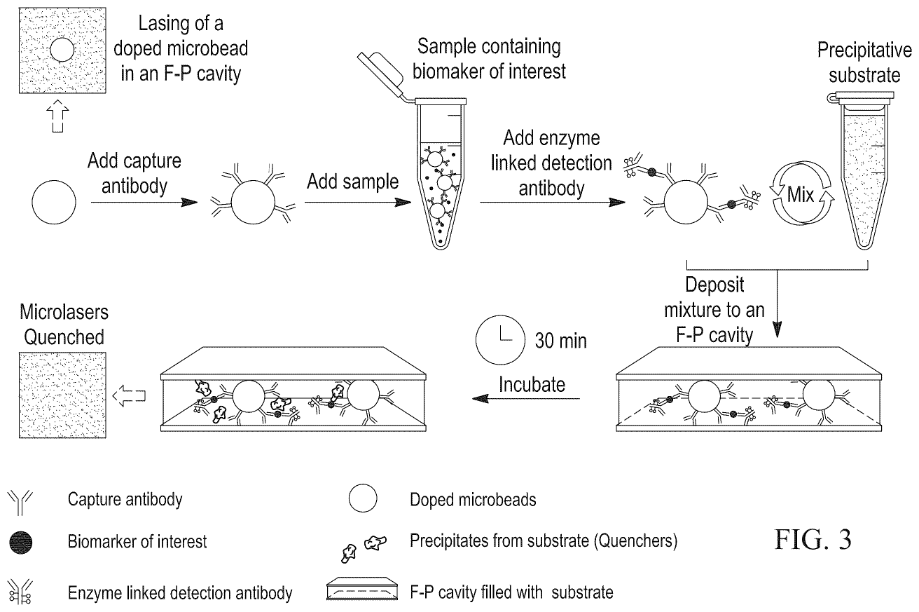

A bioassay device having a microlaser ensemble (ME) quenching bioassay protocol is shown in FIG. 3. As shown in a top panel of FIG. 3, before the microbeads are placed into a Fabry-Pérot (F-P) cavity to form the ME, they first underwent a surface modification step, an analyte capture step, and a labeling step. First, dye-doped microbeads (mean diameter: 4 mm, FCDG008, Bangs Laboratories Inc., USA) are surface modified and conjugated with the desired capture antibodies using a conjugation kit (PL01N, Bangs Laboratories Inc., USA) and following manufacturer's protocols. The microbeads were then incubated with SuperBlock™ (37515, Thermo Fisher, USA) for 60 minutes followed by two repeated wash (PBS) steps. The microbeads were gently rotated during each incubation step using a rotator (R2020, Benchmark Scientific, USA). For the analyte capture step, the test sample was mixed with functionalized microbeads and incubated for 60 minutes followed by two repeated wash (PBS) steps. For the labeling step, the microbeads were incubated with biotinylated detection antibodies for 60 minutes followed by two repeated wash (PBS) steps, and then with 1:250 diluted streptavidin poly-HRP (21140, Thermo Fisher, USA) for 30 min followed by three repeated wash steps (PBS). The microbead suspension was centrifuged at 400 g for 15 minutes to complete liquid exchange. For IL-6 ELISA experiments, antibody sets from a test kit were used (DY206, R&D Systems, USA).

Finally, these microbeads, now with captured analytes and labeled with poly-HRP, were mixed with precipitative substrate solution and immediately loaded into an F-P cavity to form the ME (see the lower panel of FIG. 3). The F-P cavity includes two highly reflective mirrors (Evaporated Coatings, Inc, USA), each of 1 inch by 1 inch in size. The distance of the F-P cavity was supported and controlled by the dye-doped microbeads (i.e., 4 mm). A 1:1 mixture of 10× concentrated metal-enhanced DAB (34065, Thermo Fisher, USA) and concentrated hydrogen peroxide (34062, Thermo Fisher, USA) was used as the substrate in this procedure. The mixture was incubated in the F-P cavity for 30 minutes so that abundant precipitates that act as quenchers for microlasers could form. Although for each analyte concentration, approximately 10,000 microbeads were used to incubate with the sample and subsequently loaded into the F-P cavity, in the measurement and data analysis step, only about 1,000 microunits located at the center of the F-P cavity were processed. Therefore, the number of microunits/microlasers in an ME was 1,000.

A simpler biotin-streptavidin binding assay is also used to evaluate the microlaser ensemble quenching based bioassay according to certain aspects of the present disclosure. In this simplified assay, biotinylated antibodies (MQ2-39C3, Thermo Fisher, USA) were conjugated to the surface of the microbeads using the same conjugation kit mentioned previously (PL01N, Bangs Laboratories Inc., USA). Streptavidin conjugated with poly-HRP served as the analyte, which was captured by biotin on microbeads through 20 minutes of incubation. The microbeads were then washed three times and mixed with the substrate solution in the same manner as the standard ME quenching immunoassay protocol described above, before they were loaded into an F-P cavity.

Lasing threshold measurements of the bioassay microunit ensemble (ME) (e.g., a Fabry-Pérot resonator cavity with microbeads and quenchers in it) was placed under a custom-built laser emission microscope where an external nanosecond laser source (473 nm, 5 kHz repletion rate) was used to scan over the microlasers, and the images were taken. Details of the laser emission microscope can be found in Q. Chen, et al., “An integrated microwell array platform for cell lasing analysis,” Lab on a Chip 17, 2814-2820 (2017), the relevant portions of which are incorporated herein by reference. A white light image of the microbeads within the F-P cavity was taken to exactly count the total number of microlasers. The ME bioassay was pumped at six pumping energy densities (25 microjoules/mm², 45 microjoules/mm², 90 microjoules/mm², 125 microjoules/mm², 250 microjoules/mm², and 400 microjoules/mm²) and the corresponding lasing fraction of the ME for each pumping energy density was recorded.

First, the quenching of microlasers by enzyme-substrate reactions was investigated and the assay protocol optimized to determine the enzyme-substrate incubation time. A single biotin-streptavidin binding assay using a fixed 1 ng/mL of streptavidin poly-HRP was performed. The four sets of MEs were prepared in the same manner according to the assay procedures described above, except that incubation time with substrate was chosen to be 0 minutes, 10 minutes, 20 minutes, and 30 minutes, respectively. Each set of the ME was scanned at six selected pumping energy densities.

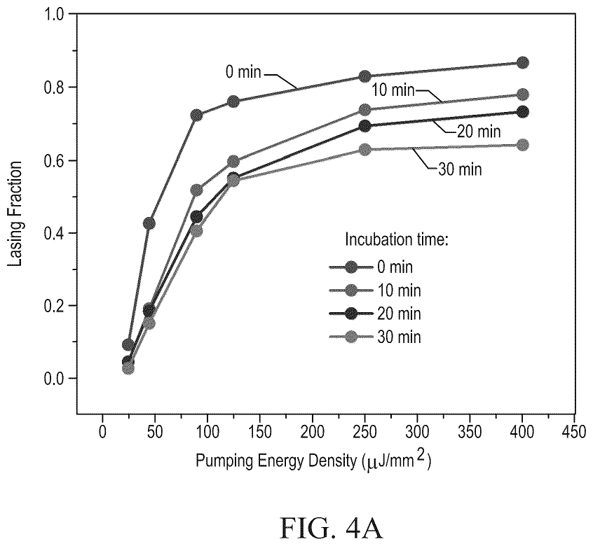

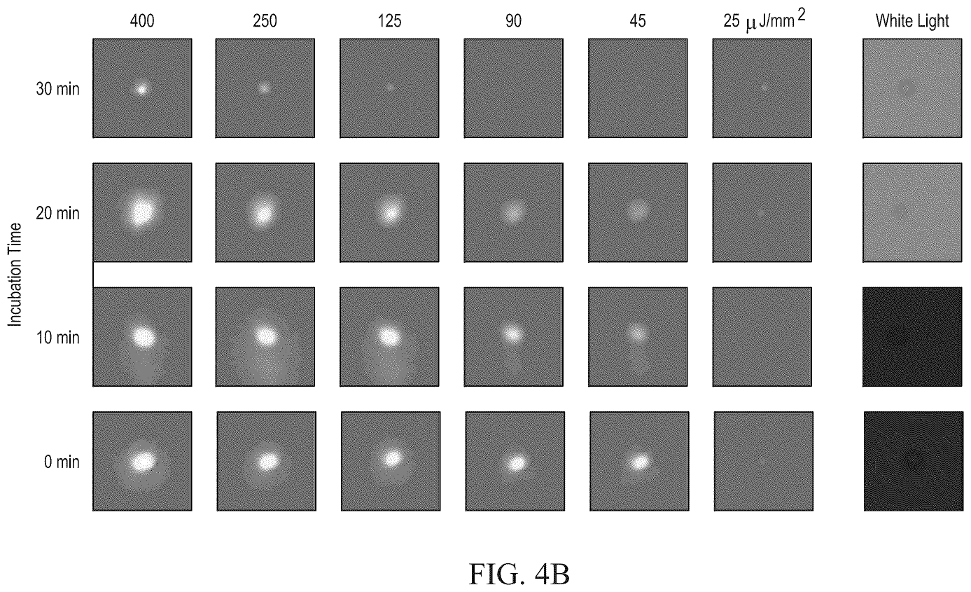

FIG. 4A shows an exemplary microlaser from each set of the ME with a fixed incubation time, illustrating how the increased incubation time affects the microlasers. As incubation time increases from 0 minutes (bottom row) to 30 minutes (top row), the microlasers' lasing thresholds increase progressively, meaning that they may be quenched more easily at a lower pumping energy density. FIG. 4B provides more quantitative analysis of the four sets of the MEs. It shows that the MEs with longer incubation times exhibit lower lasing fractions, as more quenchers can form. In subsequent experiments, an incubation time of 30 minutes is selected to achieve a strong quenching effect. Note that the incubation time here refers to the enzyme-substrate reaction time, which is an additional period on top of sample loading and other procedure-related time during the experiment.

From FIG. 4B, it can be observed that the lasing fraction is lower than 100%, even at a high pumping energy density (400 microjoules/mm²), which may suggest that there may be less effective microlasers whose gains cannot overcome the cavity loss and/or the loss caused by quenchers. While not limiting to any particular theory, it is speculated that this could be due to some microbeads having very low dye doping density of whose shape deviates so far from spherical that is causes a higher cavity loss. These microlasers are less effective (i.e., those that do not lase at the highest pumper energy density, 400 microjoules/mm² used here) will be removed in data analysis in the next section by normalization.

An ME quenching bioassay using a biotin-streptavidin binding assay is also tested. The protocol is a simplified version of immunoassays described in the above. After conjugation of biotinylated antibodies, the microbeads were separated into 13 equal portions, including 12 positive samples, incubated in 12 five-fold, serial-diluted streptavidin poly-HRP solutions and 1 negative control sample (containing 0 pg/mL streptavidin poly-HRP). For each of the 13 portions of microbeads, a corresponding set of ME is created by placing the microbeads in an F-P cavity.

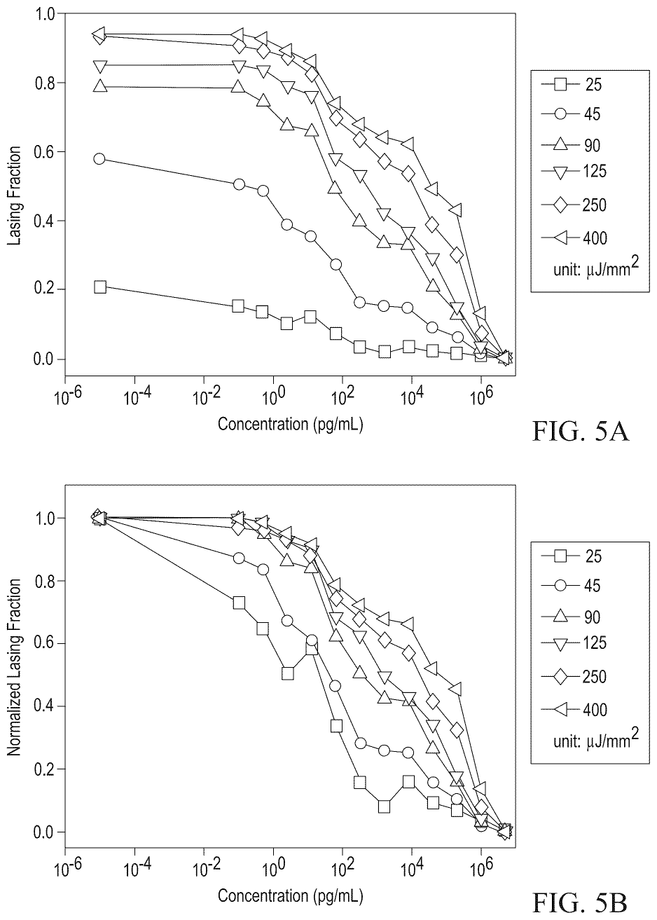

FIG. 5A shows the lasing fraction of the 13 sets of MEs under various pumping energy densities. For a given set of ME that corresponds to a fixed analyte concentration, the lasing fraction increases with the increased pumping energy density. If these datapoints are re-grouped based on each pumping energy density, which are plotted in six colored curves, within each curve (i.e., under the same pumping energy density) the lasing fraction decreases with the increased analyte concentration and hence increased number of quenchers associated with the microlasers. As discussed in FIGS. 4A-4B, there exist less effective microlasers that may not lase, even at the highest pumping energy density (400 microjoules/mm² here) that may affect a lasing fraction calculation. To remove the impact of these less effective microlasers on the lasing fraction calculation, in FIG. 5B, each lasing fraction curve is normalized to the corresponding lasing fraction obtained with the negative control, that is, when the analyte (streptavidin) concentration is 0 pg/mL (or 10⁻⁵ pg/mL in the logarithmic scale). The normalized lasing fraction data is used herein.

From FIG. 5B, it can be observed that when those microunits/microlasers are incubated with analytes of various concentrations, the (normalized) lasing fraction curves that originally converge at 0 pg/mL start to diverge. For example, at 0.1 pg/mL, the lasing fraction is about 73% for the pumping energy density of 25 microjoules/mm², whereas the lasing fraction remains nearly 100% for the pumping energy density of 400 microjoules/mm², which indicates that the number of quenchers (hence the number of analytes) varies from one microlaser to another, which results in a lasing threshold distribution among the microlasers. When different pumping energy densities are used for a given analyte concentration, the analyte distribution on the ME is probed as previously discussed above. In a hypothetical scenario in which the number of quenchers (or the number of analytes) is the same for all microlasers, the lasing fraction curves should collapse to a single curve (i.e., all curves are completely overlapped).

The lasing fraction curves in FIG. 5B can be treated as a group of calibration curves analogous to the calibration curve used in the conventional ELISA. The differences are that in the present methods, there are multiple calibration curves (six in FIG. 5B), whereas in the conventional ELISA, only one calibration curve is available. Note that in conventional fluorescence-based immunoassays, multiple calibration curves can be generated via different pumping power as well. However, due to the linear nature of the fluorescence intensity with respect to the pumping power, all calibration curves provide the same information and can be reduced to a single calibration curve after linear rescaling. In contrast, due to the nonlinear nature of the laser that has threshold behavior, the calibration curves in FIG. 5B are linearly independent and fundamentally determined by the analyte distribution on the microunits/microlasers. The calibration curves of large pumping energy densities appear to be more sensitive at high analyte concentrations, whereas the calibration curve of small pumping energy densities appear to be more sensitive at low analyte concentrations. These multiple calibration curves work together to help pinpoint the analyte concentration more precisely and enable a larger dynamic range than a single curve in the conventional ELISA.

Analyte concentration recovery using the MQE presented in Eq. (1) and the Gaussian distribution of the quenchers (analytes) on microlasers establish the connection between the lasing fraction and three variables: the pumping energy density and the two Gaussian parameters (the mean value mu (μ) and the standard deviation sigma (σ)) of the distribution of the analytes on the ME, which is reflected in Eq. (2). Furthermore, the average number of the analytes in the ME, u, is positively related to the analyte concentration in solution (C), which is similar to digital ELISA where the average number of the bright microunits is related to the analyte concentration in solution. Since u is an intermediate parameter, eventually, the relationship among the lasing fraction, pumping energy density, and the analyte concentration C, which parameters are all experimentally measurable, can be established by calibration curves obtained experimentally using function fitting of lasing fraction of the ME with respect to the experimental pumping energy density using Eq. (2).

In the fitting process, the three constants in Eq. (2), E, I₀, and Iₙₒᵣₘ, are held constant for each set of ME data, since these constants are related to the experimental conditions such as enzyme/substrate type and microlaser properties, which are the same for all sets of MEs. For each set of ME incubated in one analyte concentration Ch, two Gaussian parameters, μₕ and σₕ can be used to describe its lasing fraction distribution. In the data fitting process, the Gaussian parameter pair for all sets of ME used in fitting (uh and on, where h runs from 1 to M—the number of the sets of the MEs used in data fitting) and the three aforementioned constants (E, I₀, and Iₙₒᵣₘ) are fitted together to minimize the overall loss for all sets of the MEs. This fitting process guarantees that each data set shares the same parameters that describes the system, namely, E, I₀, and Iₙₒᵣₘ.

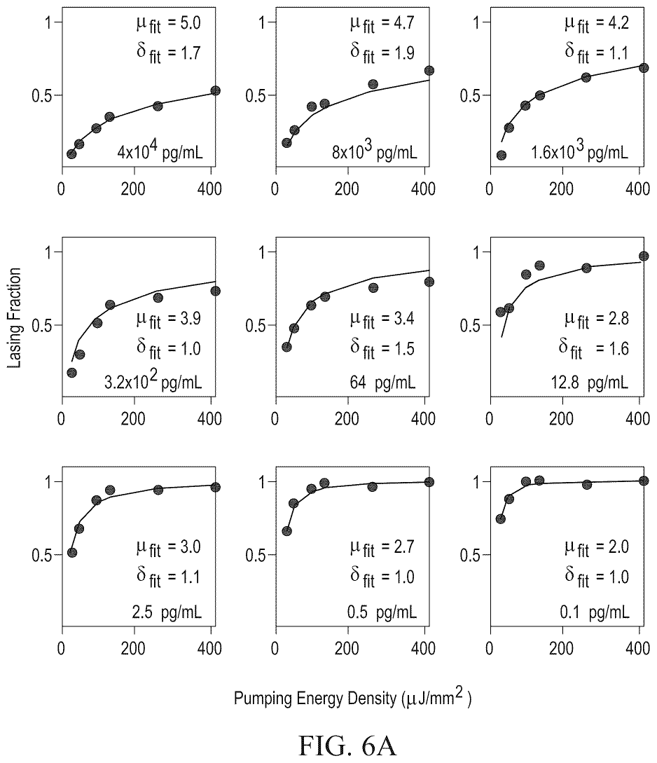

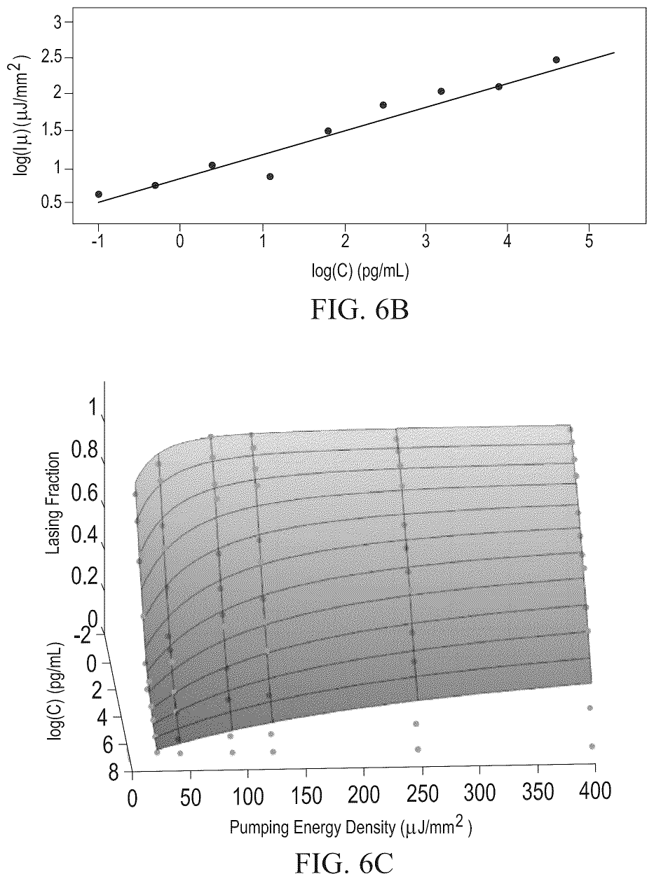

Here, the function fitting (lasing fraction versus pumping energy density) is performed for the nine sets of data (i.e., M=9) in FIG. 5B with the analyte concentration ranging from 4×10⁴ pg/mL to 1×10⁻¹ pg/mL, as shown in the subfigures of FIG. 6A along with the corresponding fitted values of μ and σ (i.e., μ_{fit} and σ_{fit}). During the above function fitting, three sets of data associated with excessively high analyte concentrations (5×10⁶ pg/mL, 1×10⁶, and 2×10⁵ pg/mL) were dropped, since their corresponding laser fractions are extremely low at all pumping energy densities and provide too few data points for a reliable Gaussian fit. For example, only one microlaser is bright in the 5×10⁶ pg/mL set of ME. Note that μ_{fit} is not necessarily the actual average number of quenchers (or analytes) of the ME, since it is subject to a rescaling factor, i.e., the fitting constant E. This is because E is an unbounded parameter and contains the information of quencher/enzyme (analyte) ratio during the incubation step. The exact value of E is determined by a microscopic quencher producing equation and it is unknown to us. However, from Eq. (2) while E is unbounded, the product, Eu, is always fixed. Therefore, depending on the value of E that is chosen, μ_{fit} varies and is rescaled by E. In this example, E is set to be one (E=1).

To quantitatively recover the analyte concentration, a new parameter—the recovered lasing threshold, I_{μ} is introduced, which is connected to μ via the following equation

$$μ = \frac{ln ( \frac{{I}_{μ} + {I}_{norm}}{{I}_{0} + {I}_{norm}} )}{E} , \qquad ( 3 )$$

$$or$$

$${I}_{μ} = ( {I}_{0} + {I}_{n o r m} ) {e}^{μ E} - {I}_{n o r m} . \qquad ( 4 )$$

Note that the invariable product of Eu is used in Eq. (4) and therefore the I_{μ} value is unique. I_{μ} can be deemed as the lasing threshold that corresponds to an imaginary microlaser with the averaged number (μ) of quenchers. FIG. 6B plots the calibration curve for I_{μ} versus the analyte concentration, showing good linearity in the logarithmic scale.

The above description provides detailed procedures of how to generate a set of calibration curves (i.e., lasing fraction vs. pumping energy density for various analyte concentrations), as shown in FIG. 6A or a single calibration curve (i.e., I_{μ} vs. analyte concentration, as shown in FIG. 6B). The above calibration curves can be used to obtain the analyte concentration for a sample under test, first the lasing fraction of an ME is measured at different pumping energy densities. Then lasing fraction versus pumping energy density will be fitted to the Gaussian cumulative distribution function, Eq. (2), where the parameter, u, can be calculated (the values of E, I₀, and Iₙₒᵣₘ will remain the same as those used during calibration curve generation). Then the calculated u will be transformed to an I_{μ} value using Eq. (4). Finally, the analyte concentration can be recovered by checking the calculated I_{μ} value with the calibration curve in FIG. 6B. Although the calibration curve in FIG. 6B looks similar to that in conventional ELISA (e.g., fluorescence signal versus analyte concentration), I_{μ} is obtained through the fitting of an ME over multiple pumping energy densities (six in this example) for significantly improved stability and robustness of measurements over single-point measurement (such as fluorescence measurement).

Based on the linearity of I_{μ} and analyte concentration in the logarithmic scale, 3D fitting of the lasing fraction versus the analyte concentration versus the pumping energy density is performed. The fitted surface is presented in FIG. 6C, showing the continuous change of the lasing fraction with respect to analyte concentration and pumping energy density. FIGS. 6B and 6C show that the system is continuously responsive over concentration change of more than five orders of magnitude and achieves a low detection limit of 0.1 pg/mL. Note that this 3D surface also works as a calibration surface (in 3D) that functions like a calibration curve in conventional ELISA (2D). The 3D calibration surface allows for pinpointing the concentration of the analyte under test using only one pumping energy density. In this case, the sample under test will incubate with an ME, then the lasing fraction will be measured at only one pumping energy density. A point on the calibration surface will be located with the measured lasing fraction under the given pumping energy density, which provides the value of the analyte concentration. In practice, even though it can be advantageous to perform multiple measurements at multiple energy densities in certain variations as discussed previously, single-point measurement also has an advantage in other variations because it is faster and simpler.

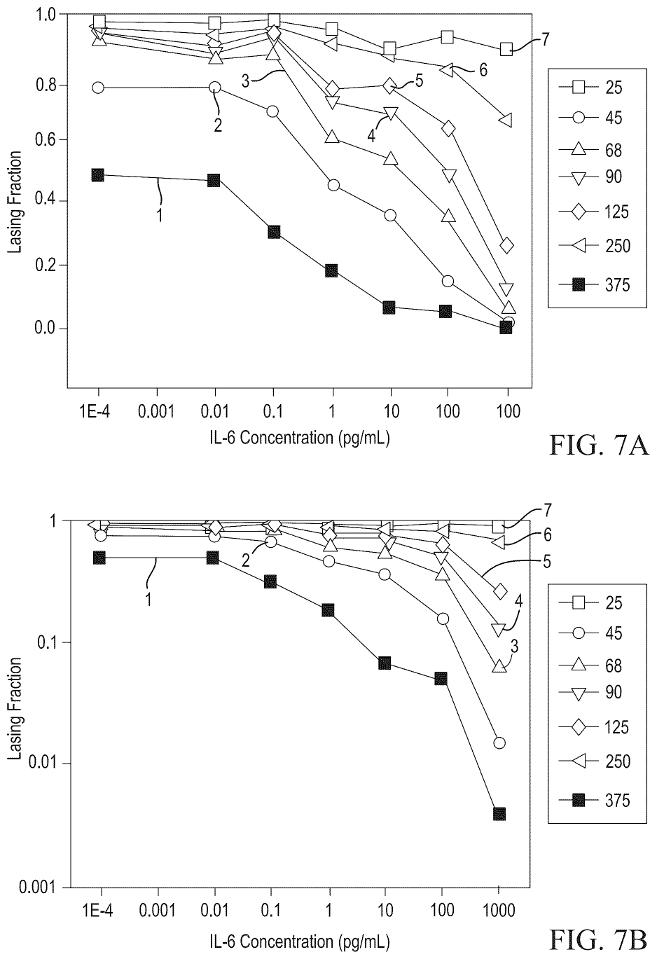

FIGS. 7A-7B shows results obtained from one example according to certain aspects of the present disclosure using IL-6 as the model target biological species analyte 22. FIG. 7A shows a fraction of lasing beads under various pumping powers in a semi-log scale, while FIG. 7B shows a log-log scale. It is observed that at a fixed pumping power from the lasing pump source 60, the fraction of the lasing beads (microlasers) decreases in general with the increased analyte concentration. However, the rate of decrease (in other words, a fraction of the lasing beads versus analyte concentration) is quite different at different pumping powers. For example, in FIG. 7A, a black curve (pumping power=25 microjoules/mm², labeled 1) decreases rapidly when the IL-6 concentration is between 0.01 pg/mL and 10 pg/mL and then the decrease rate becomes nearly zero when the IL-6 concentration is higher than 10 pg/mL. In contrast, the brown curve (pumping power=250 microjoules/mm², labeled 6) decreases slowly between 0.01 pg/mL and 1 pg/mL and then faster when the IL-6 concentration is higher than 1 pg/mL. This phenomenon is due to the nonlinear behavior of lasing emission, which does not exist when fluorescence (non-lasing emission) is used. Thus, relying on fluorescence or non-lasing light emission, such as in chemiluminescent bioassays, cannot provide the wide dynamic range of detection capabilities provided by the present technology.

Further, even considering lasing emissions, usually, the dynamic range is narrow under the same pumping power (e.g., only a single calibration curve is used). For example, in the black curve (1) in FIG. 7A, a good range of detection is between about 0.01 pg/mL and about 10 pg/mL. When the analyte concentration is higher than 10 pg/mL, the black curve (1) levels off. Therefore, it becomes difficult to tell apart 100 pg/mL and 1000 μg/mL by the fraction of the lasing beads. However, if the calibration curves obtained under different pumping powers are used together, they collectively provide a very large dynamic range. In the above example, blue (68 microjoules/mm²—labeled 3), green (90 microjoules/mm²—labeled 4), purple (125 microjoules/mm²—labeled 5), and brown curve (250 microjoules/mm²—labeled 6) to detect the analyte concentration in an overall detection range of about 10 pg/mL to about 1,000 pg/mL. Note that there is an approximate one-hundred times (100×) difference in a fraction of the lasing beads between the black curve (1) and the purple curve (125 microjoules/mm² (5), as best seen in FIG. 7B. However, in accordance with certain aspects of the present disclosure, a bioassay device can have a wide or dynamic range of detection capabilities, for example, at least one target biological species can be detected at a concentration in the fluid sample of greater than or equal to about 10 pg/mL to less than or equal to about 1,000 pg/mL (1 ng/ml).

Thus, a method is contemplated to detect and analyze a sample having an unknown concentration of analytes (such as IL-6, by way of non-limiting example). In certain variations, calibration curves are first generated under different pumping powers like those in FIGS. 7A-7B. It should be noted that if fluorescence is used as the signal, rather than lasing emissions as the signal in accordance with various aspects of the present disclosure, then the calibration curves would be the same after normalization and would not provide the advantages that the lasing emission detection does here. The sample may be run under a test and a fraction of the lasing beads under the same set of pump powers is generated. For example, seven fraction values (fraction of lasing beads) can be used at the same pumping powers as in FIGS. 7A-7B. Next, all seven values obtained are matched against the seven calibration curves in FIGS. 7A-7B. As will be appreciated by those of skill in the art, not all seven points will land on all seven calibration curves due to measurement deviations and errors. However, an algorithm can be used to provide the best match (for example, using least squares regression). In fact, using multiple points (for example, 7 points) is more robust than a single point used in regular ELISA.

Highly sensitive detection of interleukin-6 (IL-6) with a large dynamic range using a microlaser ensemble quenching based bioassay platform according to certain aspects of the present disclosure is contemplated. The assay protocol is described above. In IL-6 assay, seven sets of MEs are used, including six positive sets incubated in six ten-fold, serial-diluted IL-6 solutions (from 0.01 pg/mL to 1,000 pg/mL) and one negative control sample (containing 0 pg/mL IL-6).

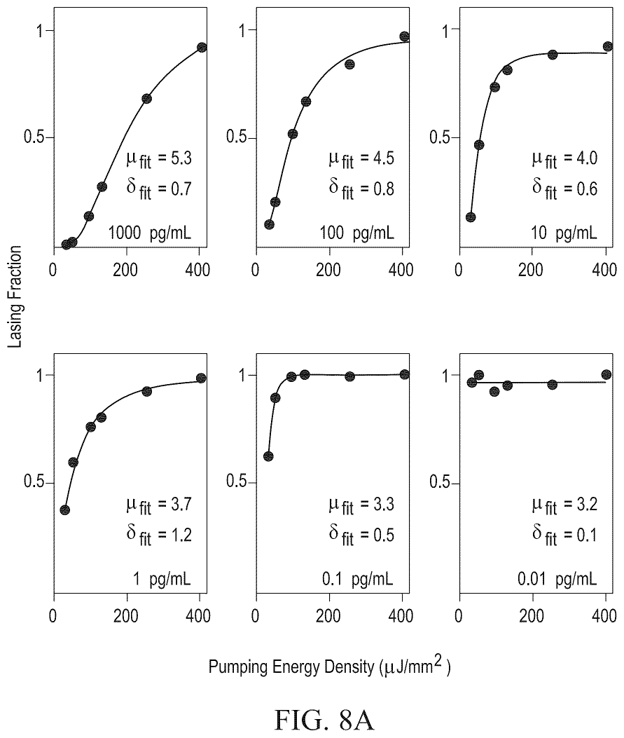

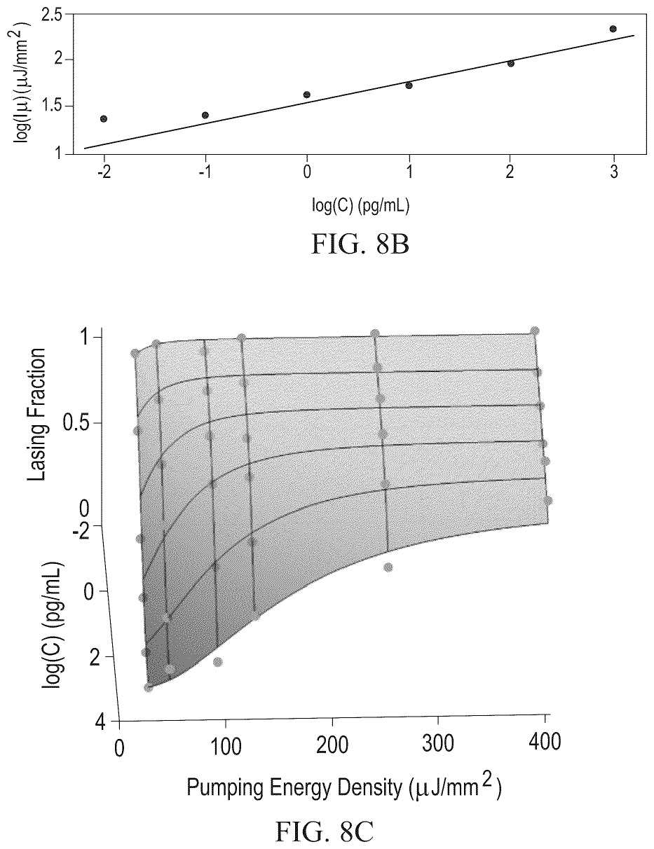

First, the function fitting of lasing fraction with respect to pumping energy density for each set of ME is presented in FIG. 8A. The fitting process is the same as the one used in FIG. 6A. Three constants E, I₀, and Iₙₒᵣₘ are held constant for all six sets of MEs. Using Eq. (5), the calibration curve of the recovered lasing threshold (I_{μ}) with respect to analyte concentration (C) can be recovered, as shown in FIG. 8B. The present methods achieves a detection limit of 0.1 pg/mL for IL-6 detection and remains sensitive at 1 ng/ml, achieving a dynamic range of four orders of magnitude.

FIG. 8C shows the 3D plot of datapoints of lasing fraction change with respect to analyte concentration and pumping energy density. The fitted surface is obtained using the linear relationship between I_{μ} and analyte concentration in the logarithmic scale in FIG. 8B. This surface shows that the inventive system is continuously responsive over four orders of magnitude. Similar to FIG. 6C, this surface plot can also function as a single-point measurement calibration surface.

Thus, as described herein, methods according to certain aspects of the present disclosure can utilize different pumping powers to generate different calibration curves that are optimized for different ranges of sensitivity. With these calibration curves, a sample having an unknown concentration of the target analyte can be run and analyzed at the same pumping powers and compared against the calibration curves to generate multiple fraction values (equal to the number of calibration curves run. For example, seven calibration curves will result in seven fraction values).

In certain other aspects, methods according to certain aspects of the present disclosure can utilize the same pumping powers, where a first pump level and a second pump level are the same. The fluid sample may be free of any target analytes at one of the first pump level or the second pump level, while the fluid sample to be analyzed for a concentration of the target analytes is analyzed at the other of the first pump level or second pump level. In this manner, a first level of lasing emissions at the first pump level and a second level of lasing emissions at a second pump level are distinct from one another, so that a concentration of the target analyte in the fluid sample can be determined by comparing each respective level of lasing emissions.

The present disclosure contemplates a new bioassay platform that uses microunits (e.g., microlaser ensembles) to probe the distribution of analytes among the microunits and subsequently deduce the analyte concentration in solution using a statistical model. This method is fundamentally different from digital ELISA. The limitation in digital detection is that while it can distinguish one analyte from zero analytes for each microunit, it is unable to differentiate one from two or more analytes in each microunit. The presence of two or more analytes in a microunit saturates the system. Therefore, digital ELISA relies on a fundamental assumption that the average number of analytes per microunit is far below one. When the average number of analytes per microunit approaches one for the entire ensemble, significant deviations arise. Consequently, digital ELISA has a limited dynamic range (especially in the upper range of detection).

In contrast, microlasers, as microunits, prepared in accordance with principles of the present disclosure have tunable dynamic ranges, which can be achieved by tuning external pumping. This concept is mathematically described in Eq. (2) above, which allows the bioassay system to achieve a large dynamic range. The current assumption in the method is that the number of analytes exceeds the number of microunits (e.g., microlaser), so that the Gaussian distribution is used. In the future, a new statistical model can be developed that may provide an even more sensitive detection and larger dynamic range for detection of even lower analyte concentrations.

Furthermore, both digital ELISA and conventional ELISA provide only one calibration curve. In digital ELISA, it is the average number of analytes versus analyte concentration. In conventional ELISA, it is detection signal (such as light intensity or absorption vs. analyte concentration) versus analyte concentration. In contrast, due to the threshold behavior intrinsic to lasers, the present methods have multiple calibration curves. By fitting multiple nonlinearly related data points to the multiple calibration curves, the methods according to certain aspects of the present disclosure significantly improve measurement stability and robustness in concentration recovery when compared to both digital and conventional ELISA.

Essentially, the methods of certain aspects of the present disclosure map the analyte distribution among detection microunits (microlasers in certain variations). This is similar to digital ELISA, which also maps the distribution of the analytes in an ensemble of microunits but at a very low average number of analytes per microunits. Alternately, conventional fluorescence based methods can be used to detect the light intensity from individual detection microunit to map the distribution, which, unlike digital ELISA, does not require the average number of analytes per microunit be far below one. However, according to the theoretical analysis conducted, microlaser quenching methods contemplated herein have a sensitivity approximately six orders of magnitude greater than that of a fluorescence based method. Therefore, the methods provided by various aspects of the present disclosure are more accurate in obtaining the analyte distribution information.

Further, use of a plurality of microunits in microlasers for a bioassay to determine the analyte concentration is distinct from previous single microlasers that use laser intensity measurement or laser onset time measurement, where these single-point measurements do not provide stable and robust data. Moreover, the laser onset time method requires extremely long assay time and the long time exposure to external pumping light may bleach the gain medium, leading to reduced sensitivity and a large variation.

In various aspects, the present disclosure contemplates methods of quantifying one or more biological species in a fluid sample. The method may comprise directing energy from a pump source at a first pump level towards a bioassay device comprising an optical cavity comprising a plurality of microunits (e.g., microlasers). As will be appreciated, the bioassay device may be any of those described previously, but will not necessarily be repeated herein for brevity. The plurality of microunits are in contact with the fluid sample potentially containing one or more biological species. The directing energy at the first pump level from the pump source generates a first level of lasing emissions. Each of the plurality of microunits comprises at least one added lasing energy responsive species that serves as a gain medium or a lossy medium to modulate lasing emissions and each microunit of the plurality of microunits comprises a targeting component capable of binding with at least one target biological species in the fluid sample. The method further includes detecting the first level of lasing emissions generated by the plurality of microunits at the first pump level. Additionally, the method may comprise directing energy from the pump source at a second pump level towards the optical cavity comprising the plurality of microunits to generate a second level of lasing emissions, followed by detecting the second level of lasing emissions generated by the plurality of microunits at the second pump level. A concentration of the at least one target biological species in the fluid sample can thus be determined by comparing the first level of lasing emissions to a first calibration curve generated at the first pump level and comparing the second level of lasing emissions to a second calibration curve generated at the second pump level.

In one aspect, the method further comprises determining the concentration in a processor, such as a computer processing unit. For example, the comparing may involve using an algorithm to compare the first level of lasing emissions to the first calibration curve and the second level of lasing emission to the second calibration curve. In certain aspects, the algorithm comprises a least squares regression analysis.

The method may further comprise detecting the at least one target biological species at a concentration of greater than or equal to about 0.01 pg/mL to less than or equal to about 1,000 pg/mL; optionally greater than or equal to about 10 pg/mL to less than or equal to about 1,000 pg/mL.

In certain aspects, the first pump level and the second pump level are the same; however, the first level of lasing emissions and the second level of lasing emissions are distinct from one another, so that the concentration of the at least one target biological species in the fluid sample can be determined by comparing each respective level of lasing emissions. For example, the method may comprise directing the energy at the first pump level or directing the energy at the second pump level towards the optical cavity, where the fluid lacks or is free of the at least one target biological species during the first pump level or the second pump level. For the other of the directing the energy at the first pump level or the second pump level, the at least one target biological species is present. For example, the method may further comprise changing at least a portion of the fluid sample (e.g., by introducing additional fluid sample to be tested or replacing an initial amount of fluid sample with a second amount of fluid sample having a distinct composition) through the optical cavity in contact with the plurality of microunits between the detecting the first level of lasing emissions generated by the plurality of microlasers at the first pump level and directing energy from the pump source at the second pump level, so that two distinct levels of lasing emissions are generated and thus the first level of lasing emissions generated by the plurality of microlasers at the first pump level and the second level of lasing emissions generated by the plurality of microlasers at the second pump level are distinct from one another. Thus, while the first pump level and the second pump level are the same, the first level of lasing emissions and the second level of lasing emissions are distinct from one another, so that the concentration of the at least one target biological species in the fluid sample can be determined by comparing each respective level of lasing emissions.

In certain aspects, the first level of lasing emissions relates to a first lasing fraction of microunits and the second level of lasing emissions relates to a second lasing fraction of microunits. In this manner, determining the concentration compares the first lasing fraction to the first calibration curve and the second lasing fraction to the second calibration curve, as was described above in the context of FIGS. 7A-7B.

Further, the directing energy from the pump source may in fact occur at a plurality of pump levels that include not only the first pump level and the second pump level, but also at least one additional pump level. Likewise, the detecting may be conducted for a plurality of levels of lasing emissions generated by the plurality of microunits. Thus, the detecting may occur for not only the first level of lasing emissions and the second level of lasing emissions, but also for at least one additional level of lasing emissions. Thus, the concentration of the at least one target biological species in the fluid sample can be determined by comparing each level of lasing emissions to a corresponding calibration curve at a respective pump level of the plurality of pump levels. In certain variations, the plurality of pump levels comprise seven distinct pump levels and the plurality of levels of lasing emissions comprise seven distinct levels of lasing emissions.

In certain aspects, the bioassay device further comprises an optical cavity configured to receive the fluid sample that optionally comprises the at least one target biological species. A plurality of microunits is disposed within the optical cavity. Each of the plurality of microunits comprises at least one added lasing energy responsive species that serves as a gain medium or a lossy medium to modulate lasing emissions when the energy is directed at the plurality of microunits. Further, each microunit of the plurality of microunits comprises a targeting component capable of binding with the at least one target biological species, so that a respective level of lasing emissions generated by the plurality of microunits relates to a quantity of the at least one target biological species in the fluid sample.

As described above, the least one added lasing energy responsive species may be a gain medium selected from the group consisting of: a dye, a quantum dot, a semiconductor material, engineered fluorescent molecules, and combinations thereof. Alternatively, the at least one added lasing energy responsive species may be a lossy medium comprising a lossy molecule or a quenching molecule.

In certain variations, the bioassay device further comprises a Fabry-Pérot resonator cavity defined between a first reflection surface and a second reflection surface. The Fabry-Pérot resonator cavity is configured to receive the fluid sample that optionally comprises the at least one target biological species. A plurality of microunits is disposed within the Fabry-Pérot resonator cavity. Each of the plurality of microunits comprises at least one added lasing energy responsive species that serves as a gain medium or a lossy medium to modulate lasing emissions when the energy is directed at the plurality of microunits. Each microunit of the plurality of microunits comprises a targeting component capable of binding with the at least one target biological species, so that a respective level of lasing emissions generated by the plurality of microunits relates to a quantity of the at least one target biological species in the fluid sample.

In certain variations, the targeting component is part of an Enzyme Linked ImmunoSorbent Assay (ELISA)-based system and the targeting component comprises a capture antibody capable of binding with the at least one target biological species. The system further comprises a detection antibody that comprises an indicator species, wherein the detection antibody further binds to the at least one target biological species that is bound to the capture antibody. The plurality of microunits may comprise a plurality of polymeric beads. Each polymeric bead defines a surface comprising at least one of the capture antibody. The plurality of polymeric beads are disposed on a substrate in the Fabry-Pérot resonator cavity. The substrate may be a metal-enhanced diaminobenzidine (DAB) peroxidase substrate. Further, the plurality of polymeric beads comprise polystyrene. As noted above, the microunits may be disposed in an array and may be present in the cavity at greater than or equal to about 1,000 microunits, optionally greater than or equal to about 1,000,000 microunits, or any of the other quantities previously articulated.

Further, as noted above, the methods of the present disclosure are not limited to the specific embodiment described above. For example, the lasing polymeric beads can be replaced with a polymer thin film, such as any polymers suitable for use as microunits. Thus, the polymer may be polystyrene (PS), by way of non-limiting example, which may optionally be mixed with a predetermined concentration of laser gain medium, such as a dye, and further treated to have a targeting component capable of binding with the at least one target biological species coated on a solid surface (such as glass slide or semiconductor wafer). The lasing energy responsive species, in the form of a gain medium, can be quantum dots or semiconductor materials (such as GaAs and InP), by way of example. These may be combined with the polymer forming a bead or film to define a composite having the quantum dot or semiconductor particles distributed therein. Further, other types of cavities such as VCSELs (vertical cavity surface emitting lasers) and ring resonators can be used. Where the system is an Enzyme Linked ImmunoSorbent Assay (ELISA)-based system, the HRP enzyme and metal enhanced DAB substrate can be replaced with other enzymes and substrates, or other types of quencher species. In addition to optical pumping, electrical pumping and other types of pump mechanisms can be used.

In one variation, the bioassay device may include an array of VCSELs with an open top mirror. Each VCSEL may be greater than or equal to about 5 micrometers to less than or equal to about 10 micrometers in diameter. The bottom mirror can be a distributed Bragg reflector (DBR) that may be used with a VCSEL and where the top mirror can be a large-sized mirror (for example, about 10 mm by about 10 mm). This top mirror is a few microns to 100 microns away from the VCSEL top surface to allow the liquid reagents to flow. All the VCSELs in the array can be electrically pumped to produce lasing emission and the pumping current can be varied. The VCSEL surface is immobilized with capture antibodies in a similar manner to the dye-doped polystyrene beads described previously in the context of FIG. 1.

The bioassay system and methods of the present disclosure can quantify a concentration of a biological target and are highly adaptable. As noted above, the microunits may take a variety of different forms. Beads can be replaced with a thin-film polymer coated on a solid surface. Further, an energy responsive species, such as a gain medium, can be a variety of distinct materials, including quantum dots or semiconductor materials.

The theory and experimental realization of the microlaser ensemble quenching bioassay platform that is fundamentally different from digital ELISA is provided herein. Using streptavidin and IL-6, this system is demonstrated to be sensitive, achieving a detection limit of 0.1 pg/mL for both, and having a dynamic range exceeding five orders of magnitude. The system uses the assumption of Gaussian distribution for analyte numbers and thus can effectively recover high analyte concentrations. The inventive platform has an advantage in diagnostic applications where test-sample biomarkers can span multiple orders of magnitude.

Expansion of the dynamic range in both the low and high end of analyte concentration is further contemplated. To detect lower concentrations of analytes, microbeads doped with a lower density of dyes can be used. Alternatively, quenchers with higher quenching strength can be used. Both approaches will make the microlaser more sensitive to the quenching effect caused by a lower number of analytes (or quenchers) associated with the microlaser(s). More homogeneous microlasers are believed to be able to reduce the lasing fraction variances in measurements, thus helping the system to reach lower concentrations. To detect higher concentrations of analytes, larger pumping energy densities (>400 microjoules/mm²) can be used to pump the ME, so that more bright microlasers can be observed to perform function fitting. Further, it is contemplated that spectral-multiplexed detection that takes advantage of narrow lasing emission spectral linewidth, which allows the detection of different lasing wavelengths from different dyes within even a single-color channel (e.g., green color). Finally, some other types of microlasers are contemplated, such as ring resonators, which do not rely on external F-P cavities, and VCSELs, which are made of semiconductor materials with high uniformity (i.e., microlaser homogeneity) and can be pumped electrically.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A bioassay device to quantify one or more biological species, the bioassay device comprising:
a region for receiving a fluid sample that optionally comprises at least one target biological species; and
a plurality of microlasers in contact with at least a portion of the fluid sample, wherein each microlaser is defined by at least one optical cavity and a microunit capable of generating lasing emissions disposed in the at least one optical cavity, wherein each microunit comprises at least one added lasing energy responsive species that serves as a gain medium or a lossy medium to modulate lasing emissions and the microunit comprises a targeting component capable of binding with the at least one target biological species in the fluid sample, so that a respective level of lasing emissions generated by the plurality of microlasers relates to an amount of energy directed at the plurality of microlasers and indicates a quantity of the at least one target biological species in the fluid sample.

2. The bioassay device of claim 1, wherein the at least one optical cavity comprises one or more of a Fabry-Pérot resonator cavity, a distributed feedback based cavity, a photonic crystal based cavity, or a ring resonator.

3. The bioassay device of claim 1, wherein each microlaser of the plurality of microlasers is a vertical cavity surface emitting laser (VCSEL), wherein the microunit comprises a cylindrical semiconductor microunit having the targeting component disposed on a surface thereof.

4. The bioassay device of claim 1, wherein the plurality of microlasers is either (i) mobile and transported through the region for receiving the fluid sample or (ii) stationary and disposed on a surface within the region for receiving the fluid sample.

5. The bioassay device of claim 1, wherein the at least one optical cavity is a single optical cavity and the plurality of microlasers is defined by a plurality of microunits disposed with the single optical cavity.

6. The bioassay device of claim 1, wherein the plurality of microlasers comprises at least 10 microlasers.

7. The bioassay device of claim 1, wherein the plurality of microlasers comprises at least 1,000 microlasers.

8. The bioassay device of claim 1, wherein the at least one added lasing energy responsive species is either:
(i) the gain medium selected from the group consisting of: a dye, a quantum dot, a semiconductor material, engineered fluorescent molecules, and combinations thereof; or
(ii) the lossy medium comprising a lossy molecule or a quenching molecule.

9. The bioassay device of claim 1, wherein the bioassay device further comprises:
a lasing pump source configured to direct the energy at the plurality of microlasers; and
a detector configured to receive and detect the lasing emissions from the plurality of microlasers.

10. A bioassay device to quantify one or more biological species, the bioassay device comprising:
a plurality of vertical cavity surface emitting lasers (VCSELs) configured to interact with a fluid sample that optionally comprises at least one target biological species, wherein the plurality of vertical cavity surface emitting lasers (VCSELs) are each associated with a targeting component capable of binding with the at least one target biological species in the fluid sample, so that a respective level of lasing emissions generated by the plurality of vertical cavity surface emitting lasers (VCSELs) relates to a quantity of the at least one target biological species in the fluid sample.

11. The bioassay device of claim 10, wherein the plurality of vertical cavity surface emitting lasers (VCSELs) each comprises a microunit comprising a semiconductor material as a gain medium.

12. The bioassay device of claim 10, wherein the targeting component is part of an Enzyme Linked ImmunoSorbent Assay (ELISA)-based system and the targeting component comprises a capture antibody capable of binding with the at least one target biological species and the Enzyme Linked ImmunoSorbent Assay (ELISA)-based system further comprises a detection antibody that comprises an indicator species, wherein the detection antibody further binds to the at least one target biological species that is bound to the capture antibody.

13. The bioassay device of claim 12, wherein a first vertical cavity surface emitting laser (VCSEL) in the plurality of vertical cavity surface emitting lasers (VCSELs) has at least one target biological species bound to the capture antibody and the detection antibody and produces a first lasing emission at a first level and a second vertical cavity surface emitting laser (VCSEL) in the plurality of vertical cavity surface emitting lasers (VCSELs) is free of any target biological species bound to the capture antibody and produces a second lasing emission at a second level, wherein the second level is greater than the first level.

14. The bioassay device of claim 13, wherein the first level relates to a quantity of the at least one target biological species present in the fluid sample.

15. The bioassay device of claim 12, wherein each vertical cavity surface emitting laser (VCSEL) of the plurality of vertical cavity surface emitting lasers (VCSELs) comprises a cylindrical semiconductor microunit that defines a surface comprising the capture antibody or a plurality of the capture antibodies.

16. The bioassay device of claim 15, wherein each cylindrical semiconductor microunit is disposed in an optical cavity that includes a metal-enhanced diaminobenzidine (DAB) peroxidase substrate.

17. The bioassay device of claim 10, wherein the plurality of vertical cavity surface emitting lasers (VCSELs) comprises at least 100 vertical cavity surface emitting lasers (VCSELs).

18. The bioassay device of claim 10, wherein the plurality of vertical cavity surface emitting lasers (VCSELs) comprises at least 1,000 vertical cavity surface emitting lasers (VCSELs).

19. The bioassay device of claim 10 having a detection range for at least one target biological species at a concentration in the fluid sample of greater than or equal to about 0.01 pg/mL to less than or equal to about 1,000 pg/mL.

20. The bioassay device of claim 10, wherein the plurality of vertical cavity surface emitting lasers (VCSELs) each comprises a microunit comprising a semiconductor material and the device further comprises:
a lasing pump source configured to direct the energy at each microunit of the plurality of vertical cavity surface emitting lasers (VCSELs) disposed in at least one optical cavity; and
a detector configured to receive and detect the lasing emissions from the plurality of vertical cavity surface emitting lasers (VCSELs).

21-34. (canceled)
